(19) United States
(12) Patent Application Publication
(10) Pub. No.: US 2021/0330518 A1
(43) Pub. Date: Oct. 28, 2021

(54) ABSORBENT ARTICLES HAVING NONWOVEN MATERIALS WITH NATURAL FIBERS

(71) Applicant: The Procter & Gamble Company, Cincinnati, OH (US)

(72) Inventors: Rodrigo ROSATI, Frankfurt Am Main (DE); Gueltekin ERDEM, Beijing (CN); Andrea L. BENNER, Schwalbach (DE)

(21) Appl. No.: 17/227,449

(22) Filed: Apr. 12, 2021

Related U.S. Application Data
(63) Continuation of application No. PCT/CN2020/086016, filed on Apr. 22, 2020.

(51) Int. Cl. A61F 13/511; A61F 13/514; A61F 13/512

(52) U.S. Cl. CPC A61F 13/51121; A61F 13/51108; A61F 2013/51083; A61F 13/512; A61F 13/51401

(57) ABSTRACT

An absorbent article comprises a liquid permeable topsheet, a liquid impermeable backsheet, and an absorbent core positioned at least partially intermediate the topsheet and the backsheet. A wearer-facing nonwoven material comprising three-dimensional features and forming at least a portion of a wearer-facing surface of the topsheet of the absorbent article is provided. The wearer-facing nonwoven material comprises a plurality of generally planar regions that do not overlap with the three-dimensional features. The generally planar regions comprise recesses. The recesses may comprise apertures. The wearer-facing nonwoven material comprises at least 50% natural fibers, by weight of the nonwoven material.

### CROSS REFERENCE TO RELATED APPLICATIONS

This application is a continuation, under 35 U.S.C. § 120, of Application No. PCT/CN2020/086016, filed on Apr. 22, 2020, which is herein incorporated by reference in its entirety.

### FIELD

The present disclosure is directed to absorbent articles comprising nonwoven materials comprising natural fibers and having reduced skin imprints.

### BACKGROUND

Absorbent articles, such as diapers, training pants, sanitary napkins, and adult incontinence products, may be used to absorb and contain urine, bowel movements, and/or menses (together “bodily exudates”). These absorbent articles may comprise nonwoven materials as various components thereof. Recently, consumers have shown interest in absorbent articles comprising nonwoven materials comprising natural fibers. These natural fibers are viewed as more environmentally friendly and of higher quality than synthetic fibers. One type of natural fiber used in nonwoven materials is cotton fibers. Nonwoven materials comprising natural fibers, however, when used as portions of wearer-facing surface of the absorbent articles, tend to cause skin imprints more than synthetic fiber nonwoven materials, especially when the nonwoven materials have three-dimensional features, recesses, and/or apertures. While skin imprints do not cause any harm to the wearer, they are not desirable to caregivers. As such, absorbent articles comprising nonwoven materials comprising natural fibers with three-dimensional features, recesses and/or apertures should be improved.

### SUMMARY

The present disclosure provides, in part, absorbent articles comprising nonwoven materials comprising natural fibers having three-dimensional features and generally planar regions. The nonwoven materials provide reduced skin imprinting of a wearer's skin. The generally planar regions may define a plurality of apertures, recesses, or recesses that define apertures. The nonwoven materials may be wearer-facing materials for skin imprint reductions, but may also be garment-facing materials or internal to absorbent articles for softness benefits. The nonwoven materials may comprise at least 15%, at least 25%, at least 50%, at least 75%, at least 80%, at least 85%, at least 90%, at least 95%, at least 99%, or even 100% natural fibers, such as cotton fibers. To solve the skin imprinting issues associated with three-dimensional, recesses, and/or apertured wearer-facing nonwoven materials comprising natural fibers, the present inventors have studied the underlying mechanisms and identified the key factors driving skin imprints. Building on this understanding, the inventors have identified a new range of patterns of three-dimensional features and/or generally planar regions (with or without apertures and/or recesses) that effectively deliver reduced skin imprints by analyzing natural fiber nonwoven materials and discovering that they are denser, stiffer, and less compliant under pressure compared to synthetic nonwoven materials. Some factors that the inventors have discovered are that the nonwoven materials comprising natural fibers should have a surface bearing area ratio %, measured under 1.86 KPa, above 45% and less than 95%, a root mean square root height (Sq), measured under 0 KPa, between about 130 microns and about 400 microns, and optionally an average area in the range of about 0.15 mm² to about 0.6 mm², preferably about 0.15 mm² to about 0.5 mm², and more preferably about 0.2 mm² to about 0.4 mm². The surface bearing area ratio % is a measure of contact between skin of a wearer and a wearer-facing nonwoven material. The greater the contact %, the more distributed localized pressure is on the skin. The root mean square root height (Sq) is an indication of the degree of three-dimensionality or texture of a nonwoven material. Higher Sq values indicate greater texture and lower Sq values indicate lower texture.

### BRIEF DESCRIPTION OF THE DRAWINGS

The above-mentioned and other features and advantages of the present disclosure, and the manner of attaining them, will become more apparent and the disclosure itself will be better understood by reference to the following description of example forms of the disclosure taken in conjunction with the accompanying drawings, wherein:

FIG. 1 is a plan view of an example absorbent article in the form of a taped diaper, garment-facing surface facing the viewer, in a flat laid-out state;

FIG. 2 is a plan view of the example absorbent article of FIG. 1, wearer-facing surface facing the viewer, in a flat laid-out state;

FIG. 3 is a front perspective view of the absorbent article of FIGS. 1 and 2 in a fastened position;

FIG. 4 is a front perspective view of an absorbent article in the form of a pant;

FIG. 5 is a rear perspective view of the absorbent article of FIG. 4;

FIG. 6 is a plan view of the absorbent article of FIG. 4, laid flat, with a garment-facing surface facing the viewer;

FIG. 7 is a cross-sectional view of the absorbent article taken about line 7-7 of FIG. 6;

FIG. 8 is a cross-sectional view of the absorbent article taken about line 8-8 of FIG. 6;

FIG. 9 is a plan view of an example absorbent core or an absorbent article;

FIG. 10 is a cross-sectional view, taken about line 10-10, of the absorbent core of FIG. 9;

FIG. 11 is a cross-sectional view, taken about line 11-11, of the absorbent core of FIG. 10;

FIG. 12 is a plan view of an example absorbent article that is a sanitary napkin;

FIG. 13 is a photograph of a nonwoven material comprising natural fibers with three-dimensional features and generally planar regions;

FIG. 14 is a plan view of a nonwoven material comprising natural fibers with three-dimensional features and generally planar regions;

FIG. 15 is a cross-sectional view of the nonwoven material of FIG. 14, taken about line 15-15 of FIG. 14;

FIG. 16 is a cross-sectional view of the nonwoven material of FIG. 14, taken about line 16-16 of FIG. 14;

FIG. 17 is a plan view of a nonwoven material comprising natural fibers with three-dimensional features and generally planar regions;

FIG. 18 is a cross-sectional view of the nonwoven material of FIG. 17, taken about line 18-18 of FIG. 17;

FIG. 19 is a cross-sectional view of the nonwoven material of FIG. 17, taken about line 19-19 of FIG. 17;

FIG. 20 is a plan view of a nonwoven material comprising natural fibers with three-dimensional features and generally planar regions;

FIG. 21 is a cross-sectional view of the nonwoven material of FIG. 20, taken about line 21-21 of FIG. 20;

FIG. 22 is a cross-sectional view of the nonwoven material of FIG. 20, taken about line 22-22 of FIG. 20;

FIG. 23 is a photograph of a nonwoven material comprising natural fibers with three-dimensional features and generally planar regions;

FIGS. 24-26 are plan views of nonwoven materials comprising natural fibers with three-dimensional features and generally planar regions;

FIG. 27 is a schematic cross-sectional illustration of a two layer nonwoven material comprising natural fibers;

FIG. 28 is a schematic cross-sectional illustration of a two layer nonwoven material comprising natural fibers with the two layers being nested with each other;

FIGS. 29 and 30 are schematic cross-sectional illustrations of two layer nonwoven materials comprising natural fibers with one layer being generally planar and the other layer having three-dimensional features and generally planar regions;

FIGS. 31 and 32 are photographs patterned nonwoven materials;

FIG. 33 is a plan view of a three-piece topsheet laminate with leg cuffs;

FIG. 34 is a cross-sectional illustration of the topsheet laminate of FIG. 33, taken about line 34-34 of FIG. 33; and

FIG. 35 is a schematic cross-sectional illustration of a bonding configuration for a three-piece topsheet laminate with leg cuffs.

### DETAILED DESCRIPTION

Various non-limiting forms of the present disclosure will now be described to provide an overall understanding of the principles of the structure, function, manufacture, and use of the absorbent articles having nonwoven materials with natural fibers disclosed herein. One or more examples of these non-limiting forms are illustrated in the accompanying drawings. Those of ordinary skill in the art will understand that the absorbent articles having nonwoven materials with natural fibers described herein and illustrated in the accompanying drawings are non-limiting example forms and that the scope of the various non-limiting forms of the present disclosure are defined solely by the claims. The features illustrated or described in connection with one non-limiting form may be combined with the features of other non-limiting forms. Such modifications and variations are intended to be included within the scope of the present disclosure.

The degree of hydrophilicity or hydrophobicity can be measured in each case by determining the contact angle of water with the specific material. The term “hydrophilic” refers to a material having a contact angle of less than or equal to 70°. The term “hydrophobic” refers to a material having a contact angle greater than 70°.

### General Description of an Absorbent Article

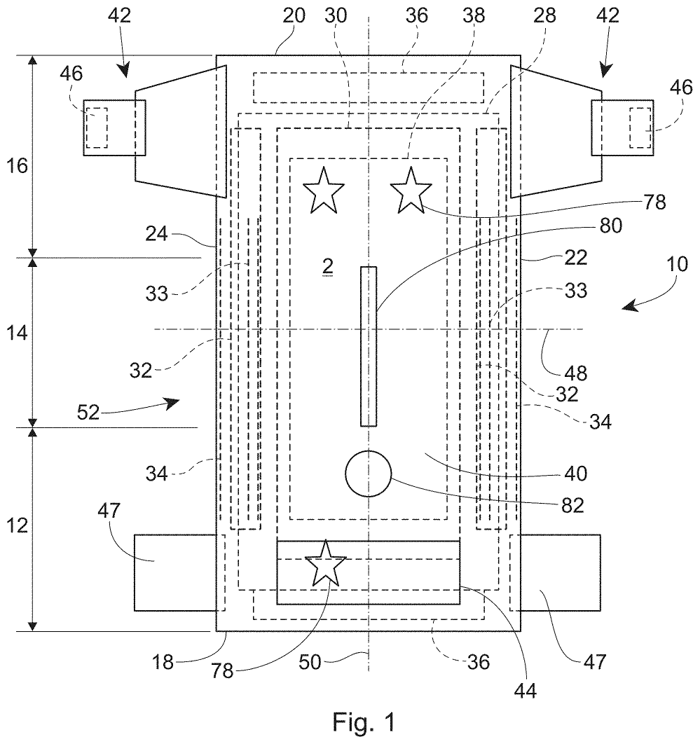

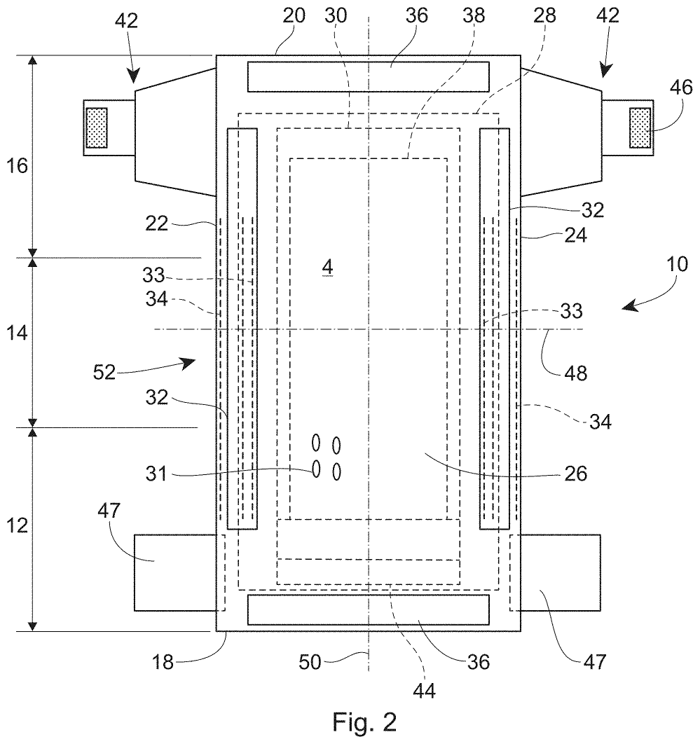

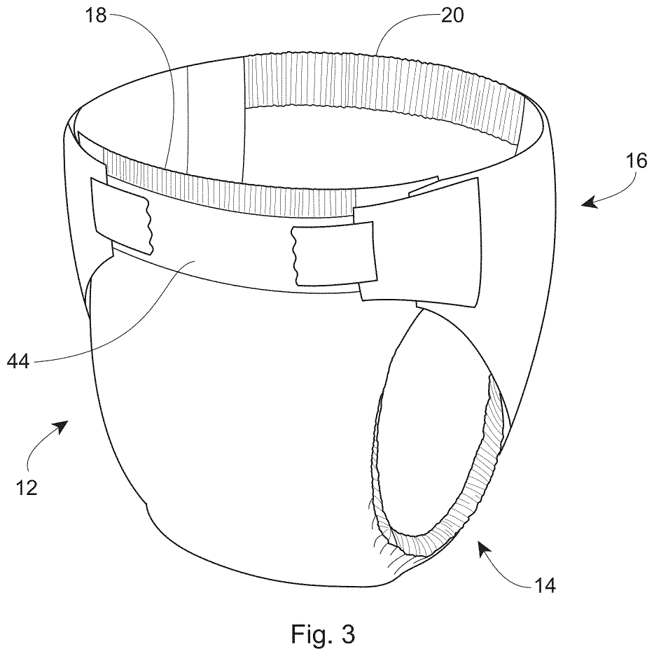

Prior to a detail description of the absorbent articles comprising nonwoven materials comprising natural fibers of the present disclosure, absorbent articles and their components will first be described. An example absorbent article 10 according to the present disclosure, shown in the form of a taped diaper, is represented in FIGS. 1-3. FIG. 1 is a plan view of the example absorbent article 10, garment-facing surface 2 facing the viewer in a flat, laid-out state (i.e., no elastic contraction). FIG. 2 is a plan view of the example absorbent article 10 of FIG. 1, wearer-facing surface 4 facing the viewer in a flat, laid-out state. FIG. 3 is a front perspective view of the absorbent article 10 of FIGS. 1 and 2 in a fastened configuration. The absorbent article 10 of FIGS. 1-3 is shown for illustration purposes only as the present disclosure may be used for making a wide variety of diapers, including adult incontinence products, pants, or other absorbent articles, such as sanitary napkins and absorbent pads, for example.

The absorbent article 10 may comprise a front waist region 12, a crotch region 14, and a back waist region 16. The crotch region 14 may extend intermediate the front waist region 12 and the back waist region 16. The front wait region 12, the crotch region 14, and the back waist region 16 may each be ⅓ of the length of the absorbent article 10. The absorbent article 10 may comprise a front end edge 18, a back end edge 20 opposite to the front end edge 18, and longitudinally extending, transversely opposed side edges 22 and 24 defined by the chassis 52.

The absorbent article 10 may comprise a liquid permeable topsheet 26, a liquid impermeable backsheet 28, and an absorbent core 30 positioned at least partially intermediate the topsheet 26 and the backsheet 28. The absorbent article 10 may also comprise one or more pairs of barrier leg cuffs 32 with or without elastics 33, one or more pairs of leg elastics 34, one or more elastic waistbands 36, and/or one or more acquisition materials 38. The acquisition material or materials 38 may be positioned intermediate the topsheet 26 and the absorbent core 30. An outer cover material 40, such as a nonwoven material, may cover a garment-facing side of the backsheet 28. The absorbent article 10 may comprise back ears 42 in the back waist region 16. The back ears 42 may comprise fasteners 46 and may extend from the back waist region 16 of the absorbent article 10 and attach (using the fasteners 46) to the landing zone area or landing zone material 44 on a garment-facing portion of the front waist region 12 of the absorbent article 10. The absorbent article 10 may also have front ears 47 in the front waist region 12. The absorbent article 10 may have a central lateral (or transverse) axis 48 and a central longitudinal axis 50. The central lateral axis 48 extends perpendicular to the central longitudinal axis 50.

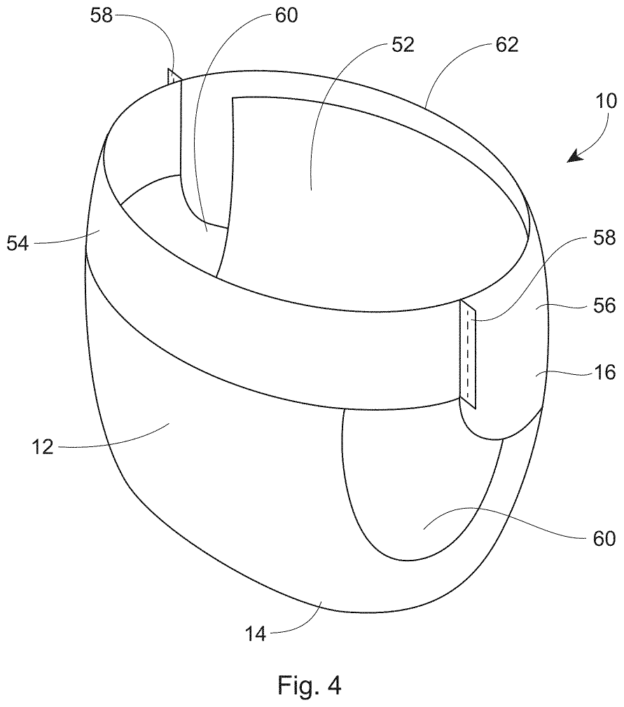

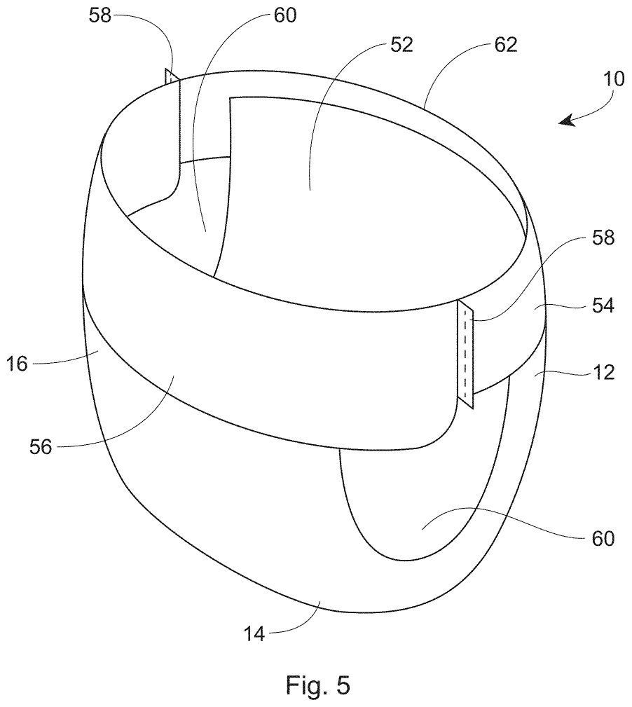

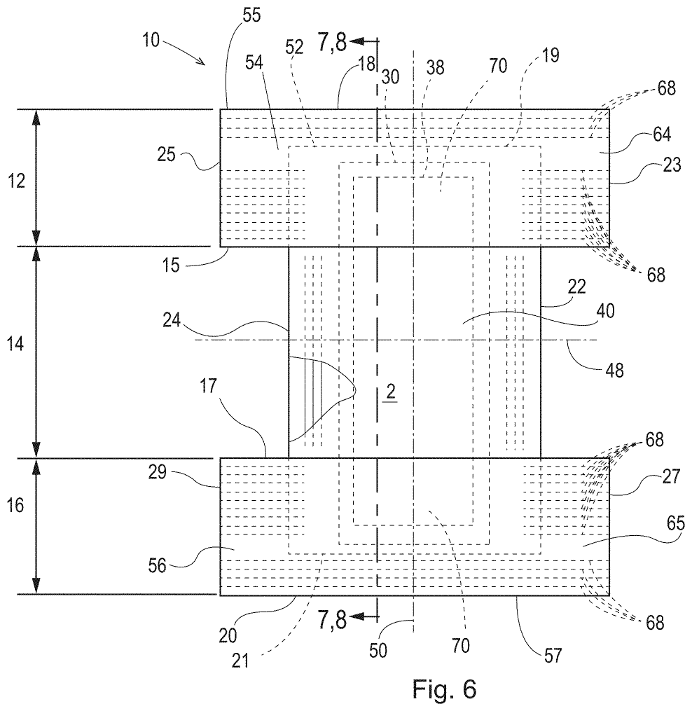

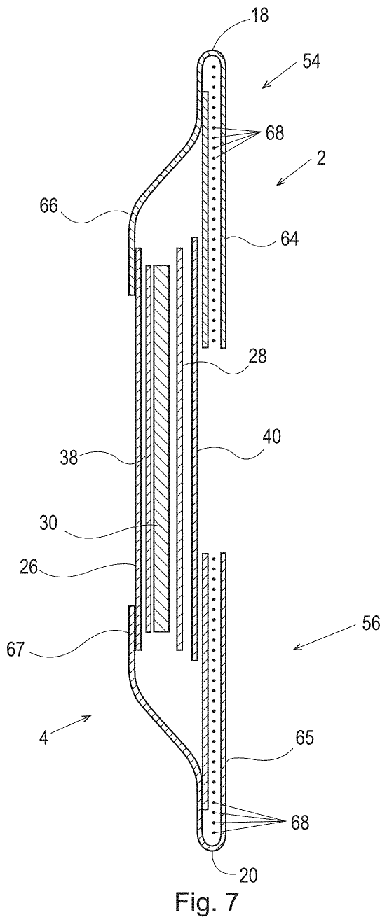

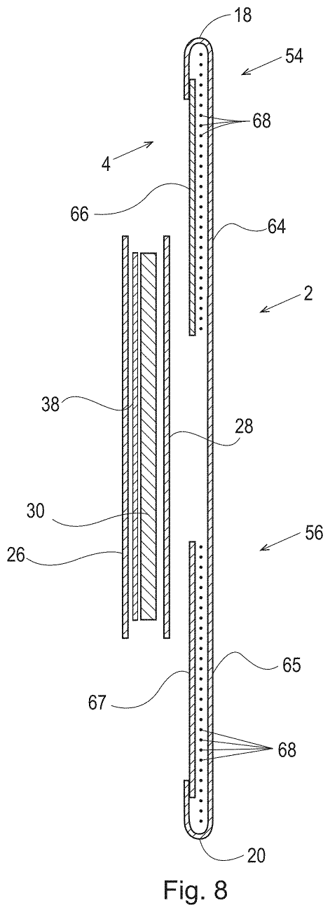

In other instances, the absorbent article may be in the form of a pant having permanent or refastenable side seams. Suitable refastenable seams are disclosed in U.S. Pat. Appl. Pub. No. 2014/0005020 and U.S. Pat. No. 9,421,137. Referring to FIGS. 4-8, an example absorbent article 10 in the form of a pant is illustrated. FIG. 4 is a front perspective view of the absorbent article 10. FIG. 5 is a rear perspective view of the absorbent article 10. FIG. 6 is a plan view of the absorbent article 10, laid flat, with the garment-facing surface facing the viewer. Elements of FIG. 4-8 having the same reference number as described above with respect to FIGS. 1-3 may be the same element (e.g., absorbent core 30). FIG. 7 is an example cross-sectional view of the absorbent article taken about line 7-7 of FIG. 6. FIG. 8 is an example cross-sectional view of the absorbent article taken about line 8-8 of FIG. 6. FIGS. 7 and 8 illustrate example forms of front and back belts 54, 56. The absorbent article 10 may have a front waist region 12, a crotch region 14, and a back waist region 16. Each of the regions 12, 14, and 16 may be ⅓ of the length of the absorbent article 10. The absorbent article 10 may have a chassis 52 (sometimes referred to as a central chassis or central panel) comprising a topsheet 26, a backsheet 28, and an absorbent core 30 disposed at least partially intermediate the topsheet 26 and the backsheet 28, and an optional acquisition material 38, similar to that as described above with respect to FIGS. 1-3. The absorbent article 10 may comprise a front belt 54 in the front waist region 12 and a back belt 56 in the back waist region 16. The chassis 52 may be joined to a wearer-facing surface 4 of the front and back belts 54, 56 or to a garment-facing surface 2 of the belts 54, 56. Side edges 23 and 25 of the front belt 54 may be joined to side edges 27 and 29, respectively, of the back belt 56 to form two side seams 58. The side seams 58 may be any suitable seams known to those of skill in the art, such as butt seams or overlap seams, for example. When the side seams 58 are permanently formed or refastenably closed, the absorbent article 10 in the form of a pant has two leg openings 60 and a waist opening circumference 62. The side seams 58 may be permanently joined using adhesives or bonds, for example, or may be refastenably closed using hook and loop fasteners, for example.

In another form, the absorbent article may be an insert for use with a reusable outer cover. The insert may be disposable or reusable. The reusable outer cover may comprise a woven or other material and may be configured as a pant or a taped diaper. In the taped context, the reusable outer cover may comprise a fastening system used to join a front waist region of the reusable outer cover to a back waist region. The fastening system may comprise snaps, buttons, and/or hooks and loops, for example. The insert may comprise a liquid permeable topsheet, a liquid impermeable backsheet, and an absorbent core positioned at least partially intermediate the topsheet and the backsheet. One or more acquisition and/or distribution materials may be positioned intermediate the topsheet and the absorbent core. The insert may comprise one or more pairs of leg cuffs and may be free of ears, side panels, and/or waistbands. In some instances, a nonwoven material may be positioned on a garment-facing side of the backsheet. A garment-facing surface of the insert may be attached to a wearer-facing surface of the reusable outer cover via adhesives, hook and loop fasteners, or other methods of joinder. An example insert and reusable outer cover system is disclosed in U.S. Pat. No. 9,011,402, issued on Apr. 21, 2015, to Roe et al. The insert or the reusable outer cover may comprise a bio-based content value from about 10% to about 100%, from about 25% to about 100%, from about 40% to about 100%, from about 50% to about 100%, from about 75% to about 100%, or from about 90% to about 100%, for example, using ASTM D6866-10, method B.

### Belts

Referring to FIGS. 7 and 8, the front and back belts 54 and 56 may comprise front and back inner belt layers 66 and 67 and front and back outer belt layers 64 and 65 having an elastomeric material (e.g., strands 68 or a film (which may be apertured)) disposed at least partially therebetween. The elastic elements 68 or the film may be relaxed (including being cut) to reduce elastic strain over the absorbent core 30 or, may alternatively, run continuously across the absorbent core 30. The elastics elements 68 may have uniform or variable spacing therebetween in any portion of the belts. The elastic elements 68 may also be pre-strained the same amount or different amounts. The front and/or back belts 54 and 56 may have one or more elastic element free zones 70 where the chassis 52 overlaps the belts 54, 56. In other instances, at least some of the elastic elements 68 may extend continuously across the chassis 52.

The front and back inner belt layers 66, 67 and the front and back outer belt layers 64, 65 may be joined using adhesives, heat bonds, pressure bonds or thermoplastic bonds. Various suitable belt layer configurations can be found in U.S. Pat. Appl. Pub. No. 2013/0211363.

Front and back belt end edges 55 and 57 may extend longitudinally beyond the front and back chassis end edges 19 and 21 (as shown in FIG. 6) or they may be co-terminus. The front and back belt side edges 23, 25, 27, and 29 may extend laterally beyond the chassis side edges 22 and 24. The front and back belts 54 and 56 may be continuous (i.e., having at least one layer that is continuous) from belt side edge to belt side edge (e.g., the transverse distances from 23 to 25 and from 27 to 29). Alternatively, the front and back belts 54 and 56 may be discontinuous from belt side edge to belt side edge (e.g., the transverse distances from 23 to 25 and 27 to 29), such that they are discrete.

As disclosed in U.S. Pat. No. 7,901,393, the longitudinal length (along the central longitudinal axis 50) of the back belt 56 may be greater than the longitudinal length of the front belt 54, and this may be particularly useful for increased buttocks coverage when the back belt 56 has a greater longitudinal length versus the front belt 54 adjacent to or immediately adjacent to the side seams 58.

The front outer belt layer 64 and the back outer belt layer 65 may be separated from each other, such that the layers are discrete or, alternatively, these layers may be continuous, such that a layer runs continuously from the front belt end edge 55 to the back belt end edge 57. This may also be true for the front and back inner belt layers 66 and 67—that is, they may also be longitudinally discrete or continuous. Further, the front and back outer belt layers 64 and 65 may be longitudinally continuous while the front and back inner belt layers 66 and 67 are longitudinally discrete, such that a gap is formed between them—a gap between the front and back inner and outer belt layers 64, 65, 66, and 67 is shown in FIG. 7 and a gap between the front and back inner belt layers 66 and 67 is shown in FIG. 8.

The front and back belts 54 and 56 may include slits, holes, and/or perforations providing increased breathability, softness, and a garment-like texture. Underwear-like appearance can be enhanced by substantially aligning the waist and leg edges at the side seams 58 (see FIGS. 4 and 5).

The front and back belts 54 and 56 may comprise graphics (see e.g., 78 of FIG. 1). The graphics may extend substantially around the entire circumference of the absorbent article 10 and may be disposed across side seams 58 and/or across proximal front and back belt seams 15 and 17; or, alternatively, adjacent to the seams 58, 15, and 17 in the manner described in U.S. Pat. No. 9,498,389 to create a more underwear-like article. The graphics may also be discontinuous.

Alternatively, instead of attaching belts 54 and 56 to the chassis 52 to form a pant, discrete side panels may be attached to side edges of the chassis 22 and 24. Suitable forms of pants comprising discrete side panels are disclosed in U.S. Pat. Nos. 6,645,190; 8,747,379; 8,372,052; 8,361,048; 6,761,711; 6,817,994; 8,007,485; 7,862,550; 6,969,377; 7,497,851; 6,849,067; 6,893,426; 6,953,452; 6,840,928; 8,579,876; 7,682,349; 7,156,833; and 7,201,744.

### Topsheet

The topsheet 26 is the part of the absorbent article 10 that is in contact with the wearer's skin. The topsheet 26 may be joined to portions of the backsheet 28, the absorbent core 30, the barrier leg cuffs 32, and/or any other layers as is known to those of ordinary skill in the art. At least a portion of, or all of, the topsheet may be liquid permeable, permitting liquid bodily exudates to readily penetrate through its thickness. The topsheet may have one or more layers. The topsheet may comprise apertures (FIG. 2, element 31), recesses, three-dimensional features, and/or may have a plurality of embossments (e.g., a bond pattern). Specific apertures, recesses, and three-dimensional features are described further herein. Any portion of the topsheet may be coated with a skin care composition, an antibacterial agent, a surfactant, and/or other beneficial agents. The topsheet may be hydrophilic or hydrophobic or may have hydrophilic and/or hydrophobic portions or layers. If the topsheet is hydrophobic, typically apertures will be present so that bodily exudates may pass through the topsheet. The nonwoven materials comprising natural fibers described herein may be used as portions of, or all of, the topsheet.

### Backsheet

The backsheet 28 is generally that portion of the absorbent article 10 positioned proximate to the garment-facing surface of the absorbent core 30. The backsheet 28 may be joined to portions of the topsheet 26, the outer cover material 40, the absorbent core 30, and/or any other layers of the absorbent article by any attachment methods known to those of skill in the art. The backsheet 28 prevents, or at least inhibits, the bodily exudates absorbed and contained in the absorbent core 10 from soiling articles such as bedsheets, undergarments, and/or clothing. The backsheet is typically liquid impermeable, or at least substantially liquid impermeable. The backsheet may, for example, be or comprise a thin plastic film, such as a thermoplastic film having a thickness of about 0.012 mm to about 0.051 mm. Other suitable backsheet materials may include breathable materials which permit vapors to escape from the absorbent article, while still preventing, or at least inhibiting, bodily exudates from passing through the backsheet.

### Outer Cover Material

The outer cover material (sometimes referred to as a backsheet nonwoven) 40 may comprise one or more nonwoven materials joined to the backsheet 28 and that covers the backsheet 28. The outer cover material 40 forms at least a portion of the garment-facing surface 2 of the absorbent article 10 and effectively “covers” the backsheet 28 so that film is not present on the garment-facing surface 2. The outer cover material 40 may comprise a bond pattern, apertures, and/or three-dimensional features. The nonwoven materials comprising natural fibers of the present disclosure may be used as a portions of, or all of, the outer cover material.

### Absorbent Core

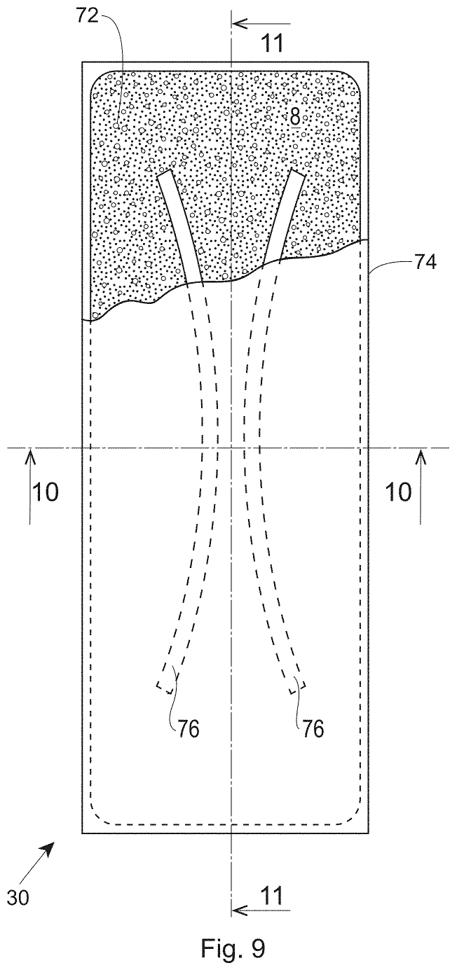

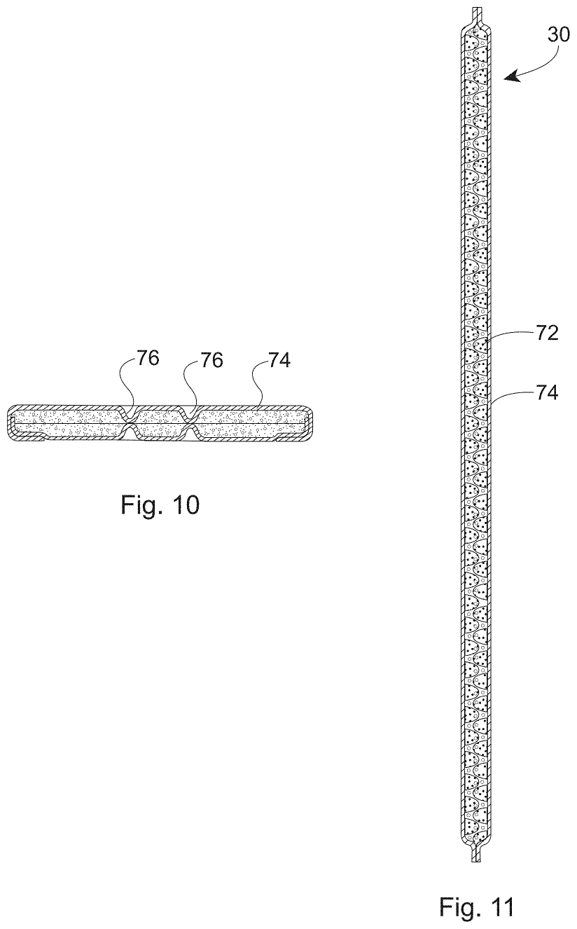

As used herein, the term “absorbent core” 30 refers to the component of the absorbent article 10 having the most absorbent capacity and that comprises an absorbent material. Referring to FIGS. 9-11, in some instances, absorbent material 72 may be positioned within a core bag or a core wrap 74. The absorbent material may be profiled or not profiled, depending on the specific absorbent article. The absorbent core 30 may comprise, consist essentially of, or consist of, a core wrap, absorbent material 72, and glue enclosed within the core wrap. The absorbent material may comprise superabsorbent polymers, a mixture of superabsorbent polymers and air felt, only air felt, and/or a high internal phase emulsion foam. In some instances, the absorbent material may comprise at least 80%, at least 85%, at least 90%, at least 95%, at least 99%, or up to 100% superabsorbent polymers, by weight of the absorbent material. In such instances, the absorbent material may be free of air felt, or at least mostly free of air felt. The absorbent core periphery, which may be the periphery of the core wrap, may define any suitable shape, such as rectangular “T,” “Y,” “hour-glass,” or “dog-bone” shaped, for example. An absorbent core periphery having a generally “dog bone” or “hour-glass” shape may taper along its width towards the crotch region 14 of the absorbent article 10.

Referring to FIGS. 9-11, the absorbent core 30 may have areas having little or no absorbent material 72, where a wearer-facing surface of the core bag 74 may be joined to a garment-facing surface of the core bag 74. These areas having little or no absorbent material and may be referred to as “channels” 76. These channels can embody any suitable shapes and any suitable number of channels may be provided. In other instances, the absorbent core may be embossed to create the impression of channels. The absorbent core in FIGS. 9-11 is merely an example absorbent core. Many other absorbent cores with or without channels are also within the scope of the present disclosure.

### Barrier Leg Cuffs/Leg Elastics

Referring to FIGS. 1 and 2, for example, the absorbent article 10 may comprise one or more pairs of barrier leg cuffs 32 and one or more pairs of leg elastics 34. The barrier leg cuffs 32 may be positioned laterally inboard of leg elastics 34. Each barrier leg cuff 32 may be formed by a piece of material which is bonded to the absorbent article 10 so it can extend upwards from a wearer-facing surface 4 of the absorbent article 10 and provide improved containment of body exudates approximately at the junction of the torso and legs of the wearer. The barrier leg cuffs 32 are delimited by a proximal edge joined directly or indirectly to the topsheet and/or the backsheet and a free terminal edge, which is intended to contact and form a seal with the wearer's skin. The barrier leg cuffs 32 may extend at least partially between the front end edge 18 and the back end edge 20 of the absorbent article 10 on opposite sides of the central longitudinal axis 50 and may be at least present in the crotch region 14. The barrier leg cuffs 32 may each comprise one or more elastics 33 (e.g., elastic strands or strips) near or at the free terminal edge. These elastics 33 cause the barrier leg cuffs 32 to help form a seal around the legs and torso of a wearer. The leg elastics 34 extend at least partially between the front end edge 18 and the back end edge 20. The leg elastics 34 essentially cause portions of the absorbent article 10 proximate to the chassis side edges 22, 24 to help form a seal around the legs of the wearer. The leg elastics 34 may extend at least within the crotch region 14. The nonwoven materials comprising natural fibers of the present disclosure may be used as a portions of the leg cuffs.

### Elastic Waistband

Referring to FIGS. 1 and 2, the absorbent article 10 may comprise one or more elastic waistbands 36. The elastic waistbands 36 may be positioned on the garment-facing surface 2 or the wearer-facing surface 4. As an example, a first elastic waistband 36 may be present in the front waist region 12 near the front belt end edge 18 and a second elastic waistband 36 may be present in the back waist region 16 near the back end edge 20. The elastic waistbands 36 may aid in sealing the absorbent article 10 around a waist of a wearer and at least inhibiting bodily exudates from escaping the absorbent article 10 through the waist opening circumference. In some instances, an elastic waistband may fully surround the waist opening circumference of an absorbent article. The nonwoven materials comprising natural fibers of the present disclosure may be used as a portions of the elastic waistband.

### Acquisition Materials

Referring to FIGS. 1, 2, 7, and 8, one or more acquisition materials 38 may be present at least partially intermediate the topsheet 26 and the absorbent core 30. The acquisition materials 38 are typically hydrophilic materials that provide significant wicking of bodily exudates. These materials may dewater the topsheet 26 and quickly move bodily exudates into the absorbent core 30. The acquisition materials 38 may comprise one or more nonwoven materials, foams, cellulosic materials, cross-linked cellulosic materials, air laid cellulosic nonwoven materials, spunlace materials, or combinations thereof, for example. In some instances, portions of the acquisition materials 38 may extend through portions of the topsheet 26, portions of the topsheet 26 may extend through portions of the acquisition materials 38, and/or the topsheet 26 may be nested with the acquisition materials 38. Typically, an acquisition material 38 may have a width and length that are smaller than the width and length of the topsheet 26. The acquisition material may be a secondary topsheet in the feminine pad context. The acquisition material may have one or more channels as described above with reference to the absorbent core 30 (including the embossed version). The channels in the acquisition material may align or not align with channels in the absorbent core 30. In an example, a first acquisition material may comprise a nonwoven material and as second acquisition material may comprise a cross-linked cellulosic material. The nonwoven materials comprising natural fibers of the present disclosure may be used as a portions of, or all of, the elastic waistband.

### Landing Zone

Referring to FIGS. 1 and 2, the absorbent article 10 may have a landing zone area 44 that is formed in a portion of the garment-facing surface 2 of the outer cover material 40. The landing zone area 44 may be in the back waist region 16 if the absorbent article 10 fastens from front to back or may be in the front waist region 12 if the absorbent article 10 fastens back to front. In some instances, the landing zone 44 may be or may comprise one or more discrete nonwoven materials that are attached to a portion of the outer cover material 40 in the front waist region 12 or the back waist region 16 depending upon whether the absorbent article fastens in the front or the back. In essence, the landing zone 44 is configured to receive the fasteners 46 and may comprise, for example, a plurality of loops configured to be engaged with, a plurality of hooks on the fasteners 46, or vice versa. The nonwoven materials comprising natural fibers of the present disclosure may be used as a portions of, or all of, the landing zones.

### Wetness Indicator/Graphics

Referring to FIG. 1, the absorbent articles 10 of the present disclosure may comprise graphics 78 and/or wetness indicators 80 that are visible from the garment-facing surface 2. The graphics 78 may be printed on the landing zone 40, the backsheet 28, and/or at other locations. The wetness indicators 80 are typically applied to the absorbent core facing side of the backsheet 28, so that they can be contacted by bodily exudates within the absorbent core 30. In some instances, the wetness indicators 80 may form portions of the graphics 78. For example, a wetness indicator may appear or disappear and create/remove a character within some graphics. In other instances, the wetness indicators 80 may coordinate (e.g., same design, same pattern, same color) or not coordinate with the graphics 78.

### Front and Back Ears

Referring to FIGS. 1 and 2, as referenced above, the absorbent article 10 may have front and/or back ears 47, 42 in a taped diaper context. Only one set of ears may be required in most taped diapers. The single set of ears may comprise fasteners 46 configured to engage the landing zone or landing zone area 44. If two sets of ears are provided, in most instances, only one set of the ears may have fasteners 46, with the other set being free of fasteners. The ears, or portions thereof, may be elastic or may have elastic panels. In an example, an elastic film or elastic strands may be positioned intermediate a first nonwoven material and a second nonwoven material. The elastic film may or may not be apertured. The ears may be shaped. The ears may be integral (e.g., extension of the outer cover material 40, the backsheet 28, and/or the topsheet 26) or may be discrete components attached to a chassis 52 of the absorbent article on a wearer-facing surface 4, on the garment-facing surface 2, or intermediate the two surfaces 4, 2. The nonwoven materials comprising natural fibers of the present disclosure may be used as a portions of the front and back ears.

### Sensors

Referring again to FIG. 1, the absorbent articles of the present disclosure may comprise a sensor system 82 for monitoring changes within the absorbent article 10. The sensor system 82 may be discrete from or integral with the absorbent article 10. The absorbent article 10 may comprise sensors that can sense various aspects of the absorbent article 10 associated with insults of bodily exudates such as urine and/or BM (e.g., the sensor system 82 may sense variations in temperature, humidity, presence of ammonia or urea, various vapor components of the exudates (urine and feces), changes in moisture vapor transmission through the absorbent articles garment-facing layer, changes in translucence of the garment-facing layer, and/or color changes through the garment-facing layer). Additionally, the sensor system 82 may sense components of urine, such as ammonia or urea and/or byproducts resulting from reactions of these components with the absorbent article 10. The sensor system 82 may sense byproducts that are produced when urine mixes with other components of the absorbent article 10 (e.g., adhesives, agm). The components or byproducts being sensed may be present as vapors that may pass through the garment-facing layer. It may also be desirable to place reactants in the absorbent article that change state (e.g. color, temperature) or create a measurable byproduct when mixed with urine or BM. The sensor system 82 may also sense changes in pH, pressure, odor, the presence of gas, blood, a chemical marker or a biological marker or combinations thereof. The sensor system 82 may have a component on or proximate to the absorbent article that transmits a signal to a receiver more distal from the absorbent article, such as an iPhone, for example. The receiver may output a result to communicate to the caregiver a condition of the absorbent article 10. In other instances, a receiver may not be provided, but instead the condition of the absorbent article 10 may be visually or audibly apparent from the sensor on the absorbent article.

### Packages

The absorbent articles of the present disclosure may be placed into packages. The packages may comprise polymeric films and/or other materials. Graphics and/or indicia relating to properties of the absorbent articles may be formed on, printed on, positioned on, and/or placed on outer portions of the packages. Each package may comprise a plurality of absorbent articles. The absorbent articles may be packed under compression so as to reduce the size of the packages, while still providing an adequate amount of absorbent articles per package. By packaging the absorbent articles under compression, caregivers can easily handle and store the packages, while also providing distribution savings to manufacturers owing to the size of the packages.

### Sanitary Napkin

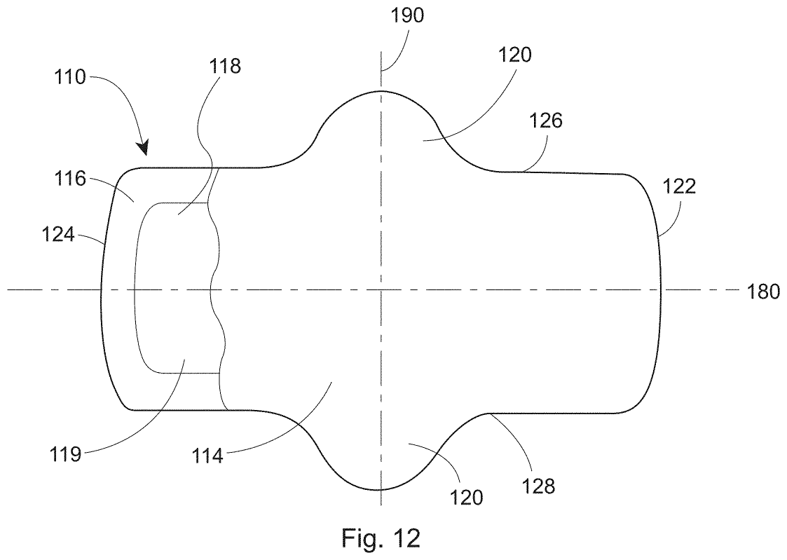

Referring to FIG. 12, an absorbent article of the present disclosure may be a sanitary napkin 110. The sanitary napkin 110 may comprise a liquid permeable topsheet 114, a liquid impermeable, or substantially liquid impermeable, backsheet 116, and an absorbent core 118. The liquid impermeable backsheet 116 may or may not be vapor permeable. The absorbent core 118 may have any or all of the features described herein with respect to the absorbent core 30 and, in some forms, may have a secondary topsheet 119 (STS) instead of the acquisition materials disclosed above. The STS 119 may comprise one or more channels, as described above (including the embossed version). In some forms, channels in the STS 119 may be aligned with channels in the absorbent core 118. The sanitary napkin 110 may also comprise wings 120 extending outwardly with respect to a longitudinal axis 180 of the sanitary napkin 110. The sanitary napkin 110 may also comprise a lateral axis 190. The wings 120 may be joined to the topsheet 114, the backsheet 116, and/or the absorbent core 118. The sanitary napkin 110 may also comprise a front edge 122, a back edge 124 longitudinally opposing the front edge 122, a first side edge 126, and a second side edge 128 longitudinally opposing the first side edge 126. The longitudinal axis 180 may extend from a midpoint of the front edge 122 to a midpoint of the back edge 124. The lateral axis 190 may extend from a midpoint of the first side edge 128 to a midpoint of the second side edge 128. The sanitary napkin 110 may also be provided with additional features commonly found in sanitary napkins as is known in the art. The nonwoven materials comprising natural fibers of the present disclosure may be used as a portions of the sanitary napkins, such as the topsheet, for example.

### Bio-Based Content for Components

Components of the absorbent articles described herein may at least partially be comprised of bio-based content as described in U.S. Pat. Appl. No. 2007/0219521A1. For example, the superabsorbent polymer component may be bio-based via their derivation from bio-based acrylic acid. Bio-based acrylic acid and methods of production are further described in U.S. Pat. Appl. Pub. No. 2007/0219521 and U.S. Pat. Nos. 8,703,450; 9,630,901 and 9,822,197. Other components, for example nonwoven and film components, may comprise bio-based polyolefin materials. Bio-based polyolefins are further discussed in U.S. Pat. Appl. Pub. Nos. 2011/0139657, 2011/0139658, 2011/0152812, and 2016/0206774, and U.S. Pat. No. 9,169,366. Example bio-based polyolefins for use in the present disclosure comprise polymers available under the designations SHA7260™, SHE150™, or SGM9450F™ (all available from Braskem S.A.). The bio-based components may comprise polylactic acid, polybutylene succinate, polyhydroxyalkanoates, bio-polypropylene, bio-polyethylene, bio-polyethylene terephthalate, carboxymethyl cellulose or starch based superabsorbent materials.

An absorbent article component may comprise a bio-based content value from about 10% to about 100%, from about 25% to about 100%, from about 40% to about 100%, from about 50% to about 100%, from about 75% to about 100%, or from about 90% to about 100%, for example, using ASTM D6866-10, method B.

### Recycle Friendly and Bio-Based Absorbent Articles

Components of the absorbent articles described herein may be recycled (e.g., chemically or mechanically) for other uses, whether they are formed, at least in part, from recyclable materials. Examples of absorbent article materials that may be recycled are nonwovens, films, fluff pulp, and superabsorbent polymers. The recycling process may use an autoclave for sterilizing the absorbent articles, after which the absorbent articles may be shredded and separated into different byproduct streams. Example byproduct streams may comprise plastic, superabsorbent polymer, and cellulose fiber, such as pulp. These byproduct streams may be used in the production of fertilizers, plastic articles of manufacture, paper products, viscose, construction materials, absorbent pads for pets or on hospital beds, and/or for other uses. Further details regarding absorbent articles that aid in recycling, designs of recycle friendly diapers, and designs of recycle friendly and bio-based component diapers, are disclosed in U.S. Pat. Appl. Publ. No. 2019/0192723, published on Jun. 27, 2019.

Absorbent Articles having Nonwoven Materials with Natural Fibers

As referenced above, absorbent articles having nonwoven materials with natural fibers are provided. The nonwoven materials may be used as portions of, or all of, topsheets, outer cover materials, leg cuffs, acquisition materials, waistbands, or other absorbent article components comprising nonwoven materials. The nonwoven materials may be wearer-facing materials for skin imprint reductions, but may also be garment-facing materials or internal to the absorbent articles for softness benefits. The nonwoven materials may comprise three-dimensional features and generally planar regions. The generally planar regions may comprise recesses, apertures, or recesses having apertures formed therein. These nonwoven materials may provide reduced skin imprinting of a wearer's skin when used a skin facing component, such as a topsheet, for example. It has been discovered that nonwoven materials formed of natural fibers, such as cotton, tend to cause more skin imprints than nonwoven materials formed of synthetic fibers, especially when the nonwoven materials have three-dimensional features, recesses, and/or apertures. While skin imprints do not cause any harm to the wearer, they are not desirable to caregivers.

The nonwoven materials may comprise at least 15%, at least 25%, at least 50%, at least 75%, at least 80%, at least 85%, at least 90%, at least 95%, at least 99%, or even 100% natural fibers, such as cotton fibers. Natural fibers comprise fibers harvested without any post-harvest treatment step as well as those having a post-treatment step, such as, for example, washing, scouring, bleaching. Natural fibers also comprise plant-based fibers, bio-based fibers, bio-based polyolefin fibers, bio-based polyester fibers, such as PLA, or combinations thereof (hereinafter referred to as “natural fibers”). The natural fibers may comprise or be hydrophilic cotton fibers and/or hydrophobic cotton fibers. To solve the skin imprinting issues associated with three-dimensional and/or apertured wearer-facing nonwoven materials comprising natural fibers, the present inventors have studied the underlying mechanisms and identified the key factors driving skin imprints. Building on this understanding, the inventors have identified a new range of patterns of three-dimensional features and generally planar regions, with or without apertures and/or recesses, that effectively deliver reduced skin imprints by analyzing natural fiber nonwoven materials and discovering that they are denser, stiffer, and less compliant under pressure compared to synthetic nonwoven materials. Some factors that the inventors have discovered are that the nonwoven materials comprising natural fibers should have a surface bearing area ratio %, measured under 1.86 KPa, above 45% and less than 95%, a root mean square root height (Sq), measured under 0 KPa, between about 130 microns and about 400 microns, and optionally an average area in the range of about 0.15 mm² to about 0.6 mm², preferably about 0.15 mm² to about 0.5 mm², and more preferably about 0.2 mm² to about 0.4 mm². The surface bearing area ratio % is a measure of contact between skin of a wearer and a wearer-facing nonwoven material. The greater the contact %, the better the reduced localized pressure is on the skin. The root mean square root height (Sq) is an indication of the degree of three-dimensionality or texture of a nonwoven material. Higher Sq values indicate greater texture and lower Sq values indicate lower texture. Smaller apertures typically cause less skin imprints than larger apertures and create smoother nonwoven materials.

### Surface Bearing Area Ratio %

As mentioned above, the surface bearing area ratio % is a measure of contact between skin of a wearer and a wearer-facing nonwoven material. The more contact the nonwoven material has with skin of a wearer during wear, the less local pressure is exerted on a wearer's skin. This may be due, for example, to weight of the wearer lying on the nonwoven material or elastics pulling the nonwoven material against the wearer's skin being distributed across larger areas of the skin which leads to reduced localized pressure and to reduced skin imprints. The nonwoven materials comprising natural fibers of the present disclosure may have a surface bearing area ratio % in the range of about 45% to about 95%, about 50% to about 90%, about 50% to about 85%, about 55% to about 85%, about 60% to about 85%, about 60%, about 65%, about 70%, about 75%, about 80%, about 82%, or about 85%, specifically reciting all 1% increments within the specified ranges and all ranges formed therein or thereby. The Surface Bearing Area Ratio % is measured according to the Surface Test herein.

### Root Mean Square Root Height (Sq)

### Measured Under 0K Pa

As mentioned above, the root mean square root height (Sq) is an indication of the degree of three-dimensionality or texture of a nonwoven material. The lower the degree of three-dimensionality or texture is, the less skin imprinting will occur. The nonwoven materials comprising natural fibers of the present disclosure may have a root mean square root height (Sq), measured under 0K Pa, in the range of about 130 microns to about 400 microns, about 130 microns to about 350 microns, about 130 microns to about 300 microns, about 130 microns to about 250 microns, about 130 microns to about 225 microns, about 130 microns to about 210 microns, about 130 microns to about 200 microns, about 140 microns to about 210 microns, about 145 microns to about 185 microns, or about 150 microns to about 180 microns, specifically reciting all 1 micron increments within the specified ranges and all ranges formed therein or thereby. The root mean square root height (Sq), measured under 0K Pa, is measured according to the Surface Test herein.

### Measured Under 1.86K Pa

The nonwoven materials comprising natural fibers of the present disclosure may have a root mean square root height (Sq), measured under 1.86K Pa, in the range of about 50 microns to about 200 microns, about 75 microns to about 175 microns, about 75 microns to about 160 microns, about 75 microns to about 150 microns, about 80 microns to about 150 microns, about 90 microns to about 150 microns, specifically reciting all 1 micron increments within the specified ranges and all ranges formed therein or thereby. The root mean square root height (Sq), measured under 1.86K Pa, is measured according to the Surface Test herein.

### Ratio of the Root Mean Square Root Height (Sq), Measured Under 1.86K Pa, to the Root Mean Square Root Height (Sq), Measured Under 0K Pa

The ratio of the root mean square root height (Sq), measured under 1.86K Pa to the root mean square root height (Sq), measured under 0K Pa, is an indication of how compliant the nonwoven material is. The lower the ratio of the root mean square root height (Sq), measured under 1.86K Pa, to the root mean square root height (Sq), measured under 0K Pa, the easier it is for the structure to deform under pressure and thereby reduce skin imprinting. The nonwoven materials comprising natural fibers of the present disclosure may have a ratio of the root mean square root height (Sq), measured under 1.86K Pa, to the root mean square root height (Sq), measured under 0K Pa, in the range of about 0.3 to about 0.85, about 0.4 to about 0.85, about 0.4 to about 0.8, about 0.5 to about 0.8, about 0.6 to about 0.8, about 0.64, about 0.65, about 0.70, about 0.75, about 0.78, or about 0.8, specifically reciting all 0.01 increments within the specified ranges and all ranges formed therein or thereby. The ratio of the root mean square root height (Sq), measured under 1.86K Pa, to the root mean square root height (Sq), measured under 0K Pa, is measured according to the Surface Test herein.

### Aperture Dimensions

The nonwoven materials comprising natural fibers may comprise apertures in the generally planar regions. The apertures may be quite small to promote smoothness of the nonwoven materials and reduced skin imprints. In some instances, the apertures may be formed in at least some of, or all of, the recesses.

### Average Area of Apertures

At least some of, or all of, the apertures may have an average area in the range of about 0.1 mm² to about 1.5 mm², about 0.1 mm² to about 1.25 mm², about 0.1 mm² to about 1.0 mm², about 0.1 mm² to about 0.8 mm², about 0.1 mm² to about 0.6 mm², about 0.12 mm² to about 0.8 mm², about 0.12 mm² to about 0.7 mm², about 0.15 mm² to about 0.6 mm², about 0.15 mm² to about 0.5 mm², about 0.15 mm² to about 0.45 mm², about 0.2 mm² to about 0.4 mm², about 0.2 mm² to about 0.35 mm², about 0.15 mm², about 0.2 mm², about 0.25 mm², about 0.3 mm², about 3.5 mm², about 0.4 mm², about 0.45 mm², or about 0.5 mm², specifically reciting all 0.1 mm² increments within the specified ranges and all ranges formed therein or thereby. The average area of the apertures is measured according to the Aperture Test herein.

### Average Aperture Length

At least some of, or all of, the apertures may have an average aperture length in the range of about 0.3 mm to about 1.5 mm, about 0.4 mm to about 1.3 mm, about 0.4 mm to about 1.1 mm, about 0.5 mm to about 1.2 mm, about 0.5 mm to about 1.1 mm, about 0.5 mm to about 1.0 mm, about 0.6 mm to about 0.9 mm, about 0.6 mm, about 0.7 mm, about 0.8 mm, or about 0.9 mm, specifically reciting all 0.1 increments within the specified ranges and all ranges formed therein or thereby. The average aperture length of the apertures is measured according to the Aperture Test herein.

### Average Aperture Width

At least some of, or all of, the apertures may have an average aperture width in the range of about 0.3 mm to about 1.3 mm, about 0.3 mm to about 1.1 mm, about 0.3 mm to about 1.0 mm, about 0.4 mm to about 0.8 mm, about 0.4 mm to about 0.7 mm, about 0.4 mm, about 0.5 mm, about 0.6 mm, or about 0.7 mm, specifically reciting all 0.1 increments within the specified ranges and all ranges formed therein or thereby. The average aperture width of the apertures is measured according to the Aperture Test herein.

### Average Perimeter of Apertures

At least some of, or all of, the apertures may have an average perimeter in the range of about 0.8 mm to about 5 mm, about 0.9 mm to about 4 mm, about 1.0 mm to about 3.5 mm, about 1.0 mm to about 3.0 mm, about 1.0 mm to about 2.8 mm, about 1.5 mm to about 2.5 mm, about 1.75 mm to about 2.25 mm, about 1.8 mm, about 1.9 mm, about 2.0 mm, about 2.1 mm, or about 2.2 mm, specifically reciting all 0.05 increments within the specified ranges and all ranges formed therein or thereby. The average perimeter of the apertures is measured according to the Aperture Test herein.

Generally Planar Regions As mentioned above, the nonwoven materials comprising natural fibers may have generally planar regions. Generally planar means designed to be substantially flat excluding the apertures and/or recesses and taking into account manufacturing tolerances. The generally planar regions may not or do not overlap with the three-dimensional features. The generally planar regions may be continuous, substantially continuous, or may be discrete.

### Average Area

At least some of, or all of, the generally planar regions may have an average area in the range of about 10 mm² to about 150 mm², about 20 mm² to about 100 mm², about 25 mm² to about 75 mm², about 30 mm² to about 60 mm², about 35 mm² to about 55 mm², about 40 mm² to about 50 mm², about 30 mm², about 35 mm², about 40 mm², about 45 mm², about 50 mm², about 55 mm², or about 60 mm², specifically reciting all 1 mm² increments within the specified ranges and all ranges formed therein or thereby. The average area of the generally planar regions is measured according to the Aperture Test herein.

### Average Perimeter

At least some of, or all of, the generally planar regions may have an average perimeter in the range of about 10 mm to about 150 mm, about 10 mm to about 125 mm, about 10 mm to about 100 mm, about 10 mm to about 75 mm, about 15 mm to about 50 mm, about 15 mm to about 40 mm, about 15 mm to about 35 mm, about 20 mm to about 35, about 20 mm, about 25 mm, about 30 mm, or about 35 mm, specifically reciting all 1 mm increments within the specified ranges and all ranges formed therein or thereby. The average perimeter of the generally planar regions is measured according to the Aperture Test herein.

### Average Minimum Distance Between Two Closest Apertures

At least some of, or all of, the generally planar regions may have an average minimum distance between two closest apertures in the range of about 0.3 mm to about 8 mm, about 0.3 mm to about 6 mm, about 0.3 mm to about 5 mm, about 0.4 mm to about 4 mm, about 0.5 mm to about 4 mm, about 0.75 mm to about 3 mm, about 0.75 mm to about 2.5 mm, about 1 mm to about 2, about 1 mm, about 1.25 mm, about 1.5 mm, about 1.75 mm, or about 2 mm, specifically reciting all 1 mm increments within the specified ranges and all ranges formed therein or thereby. The average minimum distance between two closest apertures of the generally planar regions is measured according to the Aperture Test herein.

### Number of Apertures/Recesses in a Generally Planar Regions

At least some of, or all of, the generally planar regions may each have a number of apertures, a number of recesses, or a number of recesses that have apertures. The number of apertures, recesses, or recesses having apertures may be in the range of about 5 to about 40, about 8 to about 50, about 10 to about 40, about 10 to about 35, about 10 to about 30, about 10 to about 20, about 15 to about 30, about 20 to about 30, specifically reciting all 1 integer increments within the specified ranges and all ranges formed therein or thereby. The number of recesses or apertures may be counted after identifying a generally planar region or the number of apertures may be measured according to the Aperture Test herein.

### Average Major Dimension

At least some of, or all of, the generally planar regions may have an average major dimension in the range of about 4 mm to about 25 mm, about 5 mm to about 20 mm, about 5 mm to about 15 mm, specifically reciting all 1 mm increments within the specified ranges and all ranges formed therein or thereby. The average major dimension of the generally planar regions is measured according to the Aperture Test herein.

### Average Minor Dimension

At least some of, or all of, the generally planar regions may have an average minor dimension in the range of about 2 mm to about 20 mm, about 3 mm to about 15 mm, about 4 mm to about 10 mm, specifically reciting all 1 mm increments within the specified ranges and all ranges formed therein or thereby. The average minor dimension of the generally planar regions is measured according to the Aperture Test herein.

### Basis Weight

The nonwoven materials (whether one or more layers) comprising natural fibers may have a basis weight in the range of about 10 gsm to about 60 gsm, about 15 gsm to about 50 gsm, about 15 gsm to about 45 gsm, about 15 gsm to about 40 gsm, about 20 gsm, about 25 gsm, about 30 gsm, about 35 gsm, or about 40 gsm, specifically reciting all 1 gsm increments within the specified ranges and all ranges formed therein or thereby. Basis weight is measured according to the Basis Weight Test herein.

### Examples

As shown in Table 1, options 2 and 4 fail to deliver acceptable skin imprints compared to the control option 1. Options 5 and 6, however, did deliver acceptable skin imprints, compared to the control option 1 (made with synthetic fibers and no cotton). Without wishing to be bound by theory, the inventors believe this is due to the specific topological properties of options 5 and 6 compared to options 2 and 4.

Options 5 and 6 have a surface bearing area ratio %, measured under 1.86 KPa, larger than 45%, while options 2 and 4 have a surface bearing area ratio %, measured under 1.86 KPa, smaller than 45%. A value of 45% as a limit is taken based on option 3 which shows trends in the forearm study to leave somewhat higher skin imprints, especially after 30 seconds. Small in-use tests, however, showed results to be comparable to the control option 1.

Options 5 and 6 have a root mean square root height (Sq), measured under 0 KPa, between 130 microns and 225 microns. This means that options 5 and 6 have a significant three-dimensionality in the absence of pressure. However, under 1.86 Kpa pressure, the root mean square root height (Sq) of Options 5 and 6 is significantly reduced. A parameter is defined as the ratio between Sq under 1.86 KPa and Sq under 0 KPa. Such ratio is below 0.8 for options 5 and 6. Without wishing to be bound by theory, it is believed that the lower the ratio between Sq under 1.86 KPa and Sq under 0 KPa is, the lower the chance of leaving skin imprints.

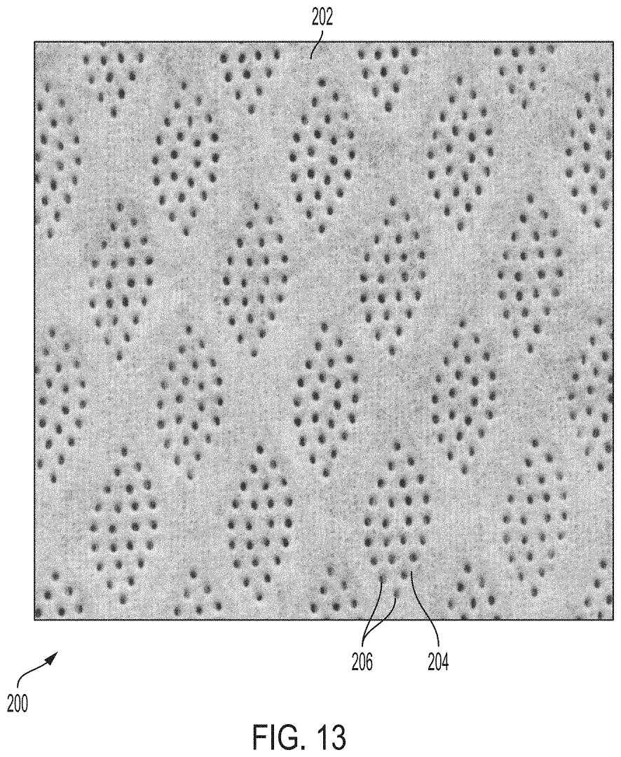

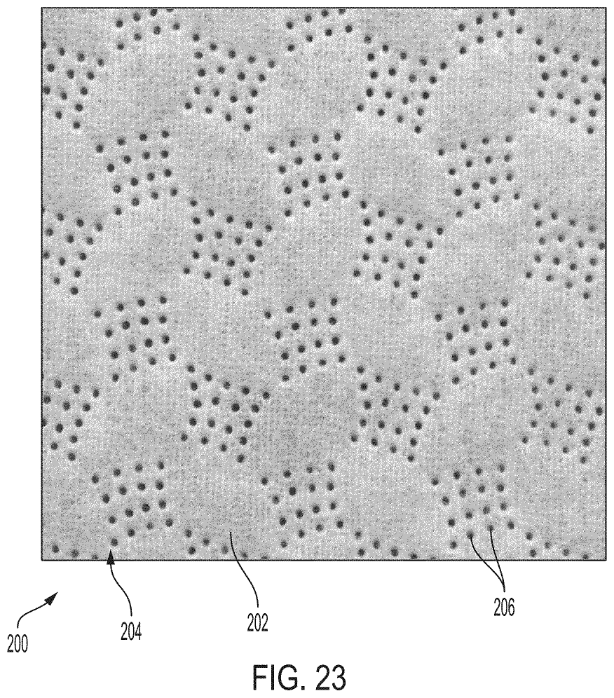

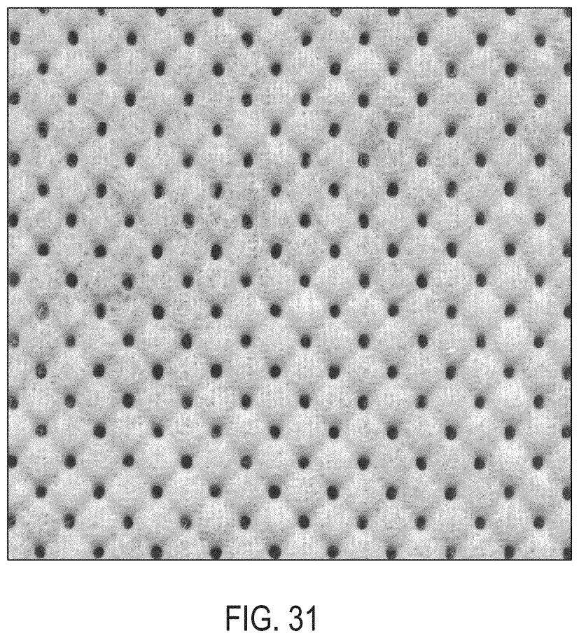

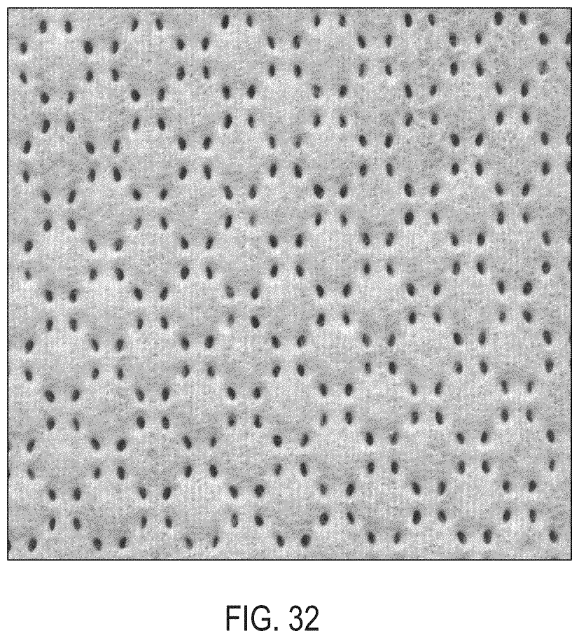

TABLE 1

| | Option | | | | | |
|---|---|---|---|---|---|---|
| | 1 | 2 | 3 | 4 | 5 | 6 |
| | (FIG. 31 | (FIG. 31 | (FIG. 32 | (FIG. 31 | (FIG. 13 | (FIG. 23 |
| Pattern of Topsheet | Pattern) | Pattern) | Pattern) | Pattern) | Pattern) | Pattern) |
| Topsheet | Control with | December | August | August | November | January |
| Description | synthetic fibers | 2017 | 2018 | 2018 | 2018 | 2019 |
| | and no cotton | Cotton | Cotton | Cotton | Cotton | Cotton |
| Top layer (wearer | Two integral carded | 35 gsm | 35 gsm | 35 gsm | 35 gsm | 35 gsm |
| facing side) | layers, 35 gsm total, | 100% | 100% | 100% | 100% | 100% |
| | top layer 15 gsm | Cotton | Cotton | Cotton | Cotton | Cotton |
| | hydrophobic PE/PET | | | | | |
| | fibers, bottom layer | | | | | |
| | 20 gsm hydrophilic | | | | | |
| | PE/PET fibers | | | | | |
| Botton layer | None | 22 gsm | 22 gsm | 22 gsm | 22 gsm | 22 gsm |
| (garmnet | | PE/PET | PE/PET | PE/PET | PE/PET | PE/PET |
| facing side) | | hydrophilic | hydrophilic | hydrophilic | hydrophilic | hydrophilic |
| | | airthrough | airthrough | airthrough | airthrough | airthrough |
| | | bonded | bonded | bonded | bonded | bonded |
| Bonding top layer to | None | xx gsm | Xx gsm | xx gsm | xx gsm | xx gsm |
| bottom layer | | glue | glue | glue | glue | glue |
| Surface bearing area | 44 (4) | 27 (5) | 45 (3) | 36 (6) | 60 (3) | 82 (5) |
| ratio % (at -100 μm | | | | | | |
| below 2%) (measured | | | | | | |
| under 1.86 KPa) | | | | | | |
| Sa (measured under | 164 (8) | 255 (12) | 158 (13) | 252 (11) | 126 (6) | 100 (8) |
| 0 KPa pressure), | | | | | | |
| micron | | | | | | |
| Sa (measured under | 74 (7) | 215 (14) | 130 (6) | 221 (8) | 95 (5) | 57 (9) |
| 1.86 KPa pressure), | | | | | | |
| micron | | | | | | |
| Sa (measured under | 0.45 | 0.84 | 0.82 | 0.88 | 0.75 | 0.57 |
| 1.86 KPa)/Sa | | | | | | |
| (measured under | | | | | | |
| 0 KPa) | | | | | | |
| Sq (measured under | 209 (10) | 333 (31) | 227 (13) | 350 (10) | 179 (4) | 152 (7) |
| 0 KPa pressure), | | | | | | |
| micron | | | | | | |
| Sq (measured under | 99 (9) | 298 (21) | 184 (4) | 309 (8) | 139 (6) | 98 (11) |
| 1.86 KPa pressure), | | | | | | |
| micron | | | | | | |
| Sq (measured under | 0.47 | 0.89 | 0.81 | 0.88 | 0.78 | 0.64 |
| 1.86 KPa)/ Sq (0 KPa) | | | | | | |
| Forearm Skin Imprint | 1/0 | 4/1.5 | 3/0.5 | 2.5/1 | 2/0 | N/A |
| Expert Grading - | | | | | | |
| 30 s/15 min (0-5) | | | | | | |
| In-use % changes w/ | 3 | 9 | 3 | 11 | 2 | N/A |
| skin imprints (All | | | | | | |
| changes) Size 4 | | | | | | |
| In-use % changes w/ | 0.3 | | | | | 0.3 |
| skin imprints (All | | | | | | |
| changes) Size 2 | | | | | | |

TABLE 2
Aperture Measurements

| Options (from Table 1) | 2 | 3 | 4 | 5 | 6 | |
|---|---|---|---|---|---|---|
| Average Aperture Area, mm\hat{ }2 | 0.71 | 0.49 | 0.86 | 0.25 (0.05) | 0.29 | (0.06) |
| Average Aperture Perimeter, mm | 3.34 | 2.82 | 3.57 | 1.91 (0.18) | 1.99 | (0.20) |
| Average Aperture Length, mm | 1.22 | 1.11 | 1.27 | 0.72 (0.07) | 0.70 | (0.06) |
| Average Aperture Width, mm | 0.78 | 0.60 | 0.90 | 0.47 (0.06) | 0.56 | (0.07) |
| Average Minimum Distance | | | | 1.60 (0.09) | 1.62 | (0.09) |
| Between Two Closest Apertures | | | | | | |
| in a generally planar region, mm | | | | | | |
| Average Area of generally | | | | 51.2 (0.0) | 39.4 | (0.0) |
| planar regions, mm\hat{ }2 | | | | | | |
| Average Perimeter of generally | | | | 30.3 (0.14) | 27.6 | (0.2) |
| planar regions,mm | | | | | | |
| Average Major Dimension of | | | | 13.5 (0.14) | 10.1 | (0.1) |
| generally planarregions, mm | | | | | | |
| Average Major Dimension of | | | | 5.65 (0.07) | 6.9 | (0.1) |
| generally planarregions, mm | | | | | | |

| | | |
|---|---|---|
| Average Number of Apertures | 24 | 19 |
| in a generallyplanar region | | |

In the forearm study the diaper, having various topsheet options of Tables 1 and 2 above, is wrapped on the forearm of a young female adult (with the topsheet facing the skin) under a pressure of 0.4 psi via using a blood pressure cuff device. After 15 minutes, the diaper is removed, skin imprints on the forearm are graded by an expert grader both at 30 seconds and at 15 minutes after removing the diaper from the forearm. Gradings range from 0 to 5, where:

| PM scale | |
|---|---|
| 0 | No skin imprints |
| 1 | Pattern partly visible |
| 2 | Pattern lightly visible |
| 3 | Pattern well visible |
| 4 | Pattern very well visible - some light skin redness |
| 5 | Pattern heavily visible including skin redness |

In order to validate the lab learnings, the inventors conducted limited in-use testing. Codes 1, 3, 4, 2, 5 were placed in a test, where panelists were asked the question at each change whether they could observe skin imprints on skin. Codes 1 and 6 were placed in an in-use test and the same question about skin imprints was asked. In-use data confirmed the lab data and forearm skin imprint data. Both codes 5 and 6 perform comparable to the control (no cotton), despite having 100% cotton layer in direct contact with the skin.

### Comparative Examples

TABLE 3

| Code | Code (7) | Code (8) | Code (9) | Code (10) | Code (11) |
|---|---|---|---|---|---|
| Topsheet Description | Flat 100% | 70% Cotton, | Flat with large | 70% viscose, | Small apertures |
| | cotton with | 20% PLA, 10% | apertures, 8mesh | 30% polyester | 100% HyDry |
| | small apertures | Tencel | 100% Cotton | with texture | Cotton |
| | 40 gsm | Hydrophobic | 35 gsm | formed by | 35 gsm |
| | COMMERCIAL | 35 gsm | COMMERCIAL | hydrojets | COMMERCIAL |
| | | COMMERCIAL | | 39 gsm | |
| Lot Number | Lot#: 06609- | Lot# n/a | Lot# n/a | Lot# n/a | Lot# 11004274 |
| | 0001~0021 | | | | |
| Country/Date of pick-up | Made in Japan, | Sample received | Made in China, | Sample | Made in France, |
| | Date 2019 | Italy, May 2017 | rolls received | received | received October |
| | | | August 2017 | summer 2019 | 2016 |
| Surface bearing area ratio % (at - | 84 (4) | 61 (21) | 36 (3) | 52 (2) | 41 (2) |
| 100 μm below 2%) (measured | | | | | |
| under 1.86 KPa) | | | | | |
| Sa (measured under 0 KPa | 58 (15) | 72 (1) | 286 (62) | 64 (7) | 260 (99) |
| pressure), micron | | | | | |
| Sa (measured under 1.86 KPa | 30 (2) | 50 (10) | 120 (12) | 45 (1) | 71 (4) |
| pressure), micron | | | | | |
| Sa (measured under 1.86 KPa)/Sa | 0.52 | 0.69 | 0.42 | 0.70 | 0.27 |
| (0 KPa) [1] | | | | | |
| Sq (measured under 0 KPa | 99 (15) | 114 (4) | 368 (94) | 88 (11) | 339 (118) |
| pressure), micron | | | | | |
| Sq (measured under 1.86 KPa | 43 (3) | 77 (21) | 144 (15) | 59 (1) | 86 (4) |
| pressure), micron | | | | | |
| Sq (measured under 1.86 KPa)/Sq | 0.43 | 0.68 | 0.39 | 0.67 | 0.25 |
| (measured under 0 Kpa) [1] | | | | | |

Examples 7, 8, 9, 11 represent competitive examples of topsheets with high cotton content. While these samples have not been tested for skin imprints with same methods for the examples of the present disclosure, these samples are outside of the claims of the present disclosure as they lack the three-dimensional features described for the nonwoven materials of the present disclosure. In addition, the competitive examples have a surface bearing area ratio % and Sq, measured under 0 KPa, outside of the claimed ranges. Furthermore, Example 10 has an Sq, measured under 0 KPa, of 88 microns, which is outside of the claimed ranges.

FIG. 13 is a photograph of an example of a nonwoven material comprising natural fibers 200. The nonwoven material 200 may be hydroentangled and may comprise natural and synthetic fibers or only natural fibers. The nonwoven material 200 may form a portion of, or all of, a topsheet, an outer cover material, an acquisition material, a landing zone, a cuff, or other component of an absorbent article. Typically, the side of the nonwoven material 200 facing the viewer in FIG. 13 will form a portion of a garment-facing surface or a wearer-facing surface depending on which component the nonwoven material 200 is used. The nonwoven material may comprise three-dimensional features 202. The three-dimensional features 202 may be continuous (as illustrated in FIG. 13), substantially continuous, or may be discrete. The three-dimensional features 202 may be formed in zones. The zones may not overlap generally planar regions. The nonwoven material 200 may comprise a plurality of generally planar regions 204. The generally planar regions 204 may be continuous or discrete (as illustrated in FIG. 13). The generally planar regions may define a plurality of apertures 206, a plurality of recesses, and/or a plurality of recesses defining apertures therein. Apertures are illustrated in the figures, but it will be understood that the recesses may be in the same locations and patterns as the apertures. The apertures or recesses may aid in bodily exudate acquisition when the nonwoven material 200 is used as a topsheet, for example. The three-dimensional features 202 may comprise protrusions that extend outwardly (out of the page) further than the generally planar regions.

The zones of the three-dimensional features may be fully or partially surrounded on at least two sides, three sides, or all sides by the generally planar regions. The generally planar regions may be fully or partially surrounded on at least two sides, three sides, or all sides by the zones of the three-dimensional features.

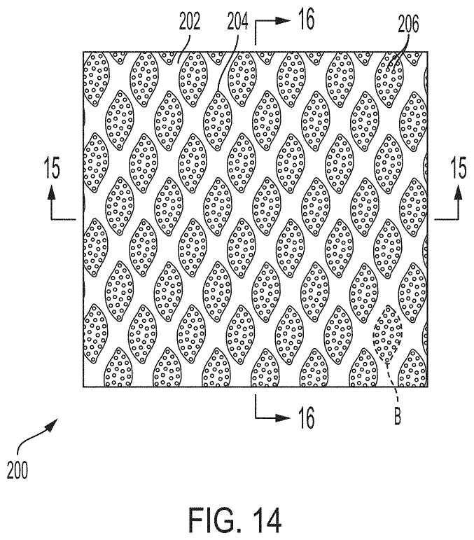

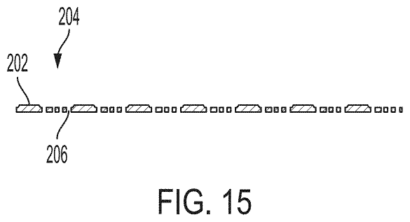

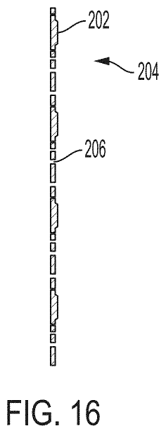

FIG. 14 is a plan view of a nonwoven material 200 comprising natural fibers of the present disclosure. The nonwoven material 200 may comprise three-dimensional features 202 and a plurality of generally planar regions 204. The generally planar regions may define a plurality of apertures 206, a plurality of recesses, and/or a plurality of recesses defining apertures therein. FIG. 15 is a cross-sectional view of the nonwoven material 200 of FIG. 14 taken about line 15-15. FIG. 16 is a cross-sectional view of the nonwoven material 200 of FIG. 14, taken about line 16-16. In FIGS. 15 and 16 it can be noted how the three-dimensional features 202 extend outwardly further than the generally planar regions. The planar nature of the generally planar regions may also be observed.

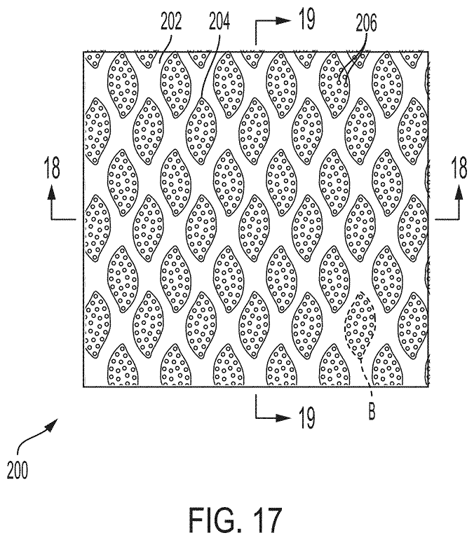

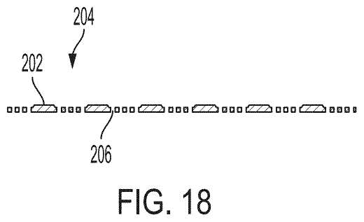

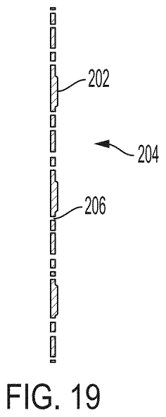

FIG. 17 is a plan view of a nonwoven material 200 comprising natural fibers of the present disclosure. The nonwoven material 200 may comprise three-dimensional features 202 and a plurality of generally planar regions 204. The generally planar regions may define a plurality of apertures 206, a plurality of recesses, and/or a plurality of recesses defining apertures therein. FIG. 18 is a cross-sectional view of the nonwoven material 200 of FIG. 17 taken about line 18-18. FIG. 19 is a cross-sectional view of the nonwoven material 200 of FIG. 17, taken about line 19-19. In FIGS. 18 and 19 it can be noted how the three-dimensional features 202 extend outwardly further than the generally planar regions. The planar nature of the generally planar regions may also be observed.

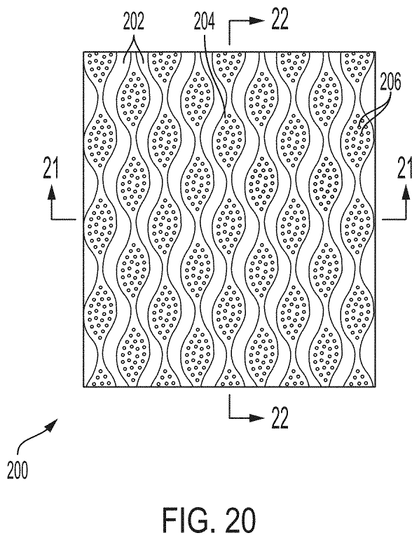

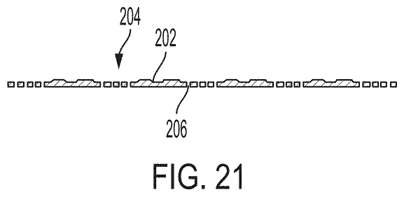

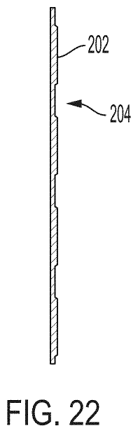

FIG. 20 is a plan view of a nonwoven material 200 comprising natural fibers of the present disclosure. The nonwoven material 200 may comprise three-dimensional features 202 and a plurality of generally planar regions 204. The generally planar regions may define a plurality of apertures 206, a plurality of recesses, and/or a plurality of recesses defining apertures therein. FIG. 21 is a cross-sectional view of the nonwoven material 200 of FIG. 20 taken about line 21-21. FIG. 22 is a cross-sectional view of the nonwoven material 200 of FIG. 20, taken about line 22-22. In FIGS. 21 and 22 it can be noted how the three-dimensional features 202 extend outwardly further than the generally planar regions. The planar nature of the generally planar regions may also be observed.

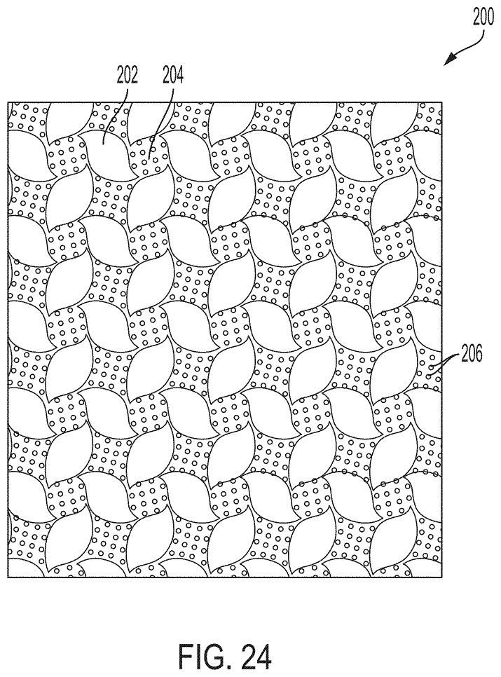

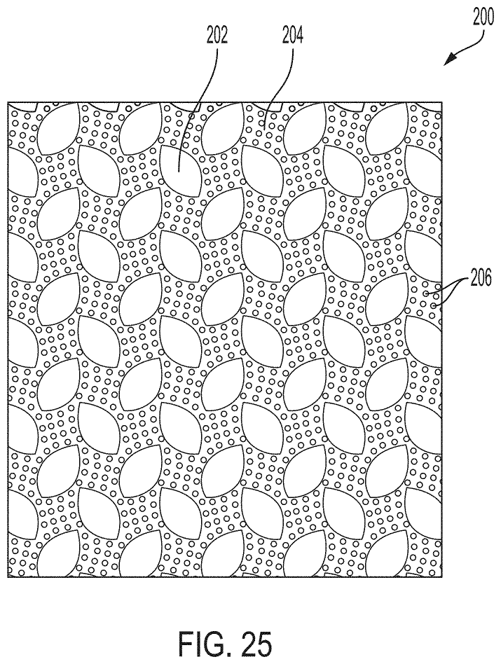

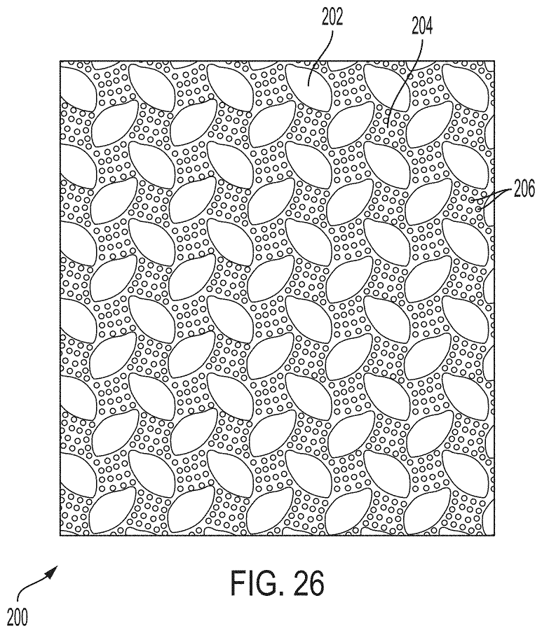

FIG. 23 is a photograph of an example of a nonwoven material comprising natural fibers 200. The nonwoven material 200 may form a portion of, or all of, a topsheet, an outer cover material, an acquisition material, a landing zone, a cuff, or other component of an absorbent article. Typically, the side of the nonwoven material 200 facing the viewer in FIG. 23 will form a portion of a garment-facing surface or a wearer-facing surface depending on which component the nonwoven material 200 is used. The nonwoven material may comprise three-dimensional features 202. The three-dimensional features 202 may be continuous (as illustrated in FIG. 23) or may be discrete. The nonwoven material 200 may comprise a plurality of generally planar regions 204. The generally planar regions 204 may be continuous or discrete (as illustrated in FIG. 23). The generally planar regions may define a plurality of apertures 206, a plurality of recesses, and/or a plurality of recesses defining apertures therein. The apertures or recesses may aid in bodily exudate acquisition when the nonwoven material is used as a topsheet, for example. The three-dimensional features 202 may comprise protrusions that extend outwardly (out of the page) further than the generally planar regions. FIGS. 24-26 are plan views of other example of nonwoven materials comprising natural fibers 200 similar to the pattern shown in FIG. 23. It is noted that in FIGS. 24-26, the generally planar regions 204 are continuous while the three-dimensional features 202 are discrete.

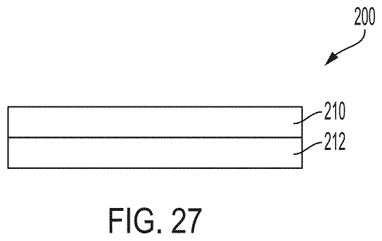

The nonwoven materials comprising natural fibers 200 may be formed of one or more layers, such as two, three, or four, for example. Both of, or all of, the layers may be formed of 100% natural fibers, such as cotton. In other instances, only one layer may be formed of 100% natural fibers. Referring to FIG. 27, as an example, a first layer 210 may be formed of 100% natural fibers and a second layer 212 may be formed of 100% natural fibers. In other instances, only one of the layers 210, 212 may be formed of natural fibers, with the other layer being formed of synthetic fibers, such as spunbond or carded polyolefin fibers. In such an instance, the synthetic fibers may comprise mono-component fibers, bi-component fibers, or multi-component fibers. The first layer 210 may be the layer that forms a portion of a garment-facing surface or a wearer-facing surface on an absorbent article. The first layer 210 may comprise up to 100% natural fibers, while the second layer 212 may comprise the synthetic fibers and may or may not be free of natural fibers. In some instances, one or more of the layers may comprise natural fibers and synthetic fibers. For example, a layer may comprise 15% to 95% natural fibers with the remainder being synthetic fibers. Any other suitable ratios of natural fibers to synthetic fibers, such as 30% natural and 70% synthetic, 40% natural and 60% synthetic, or 50% natural and 50% synthetic, are also within the scope of the present disclosure.

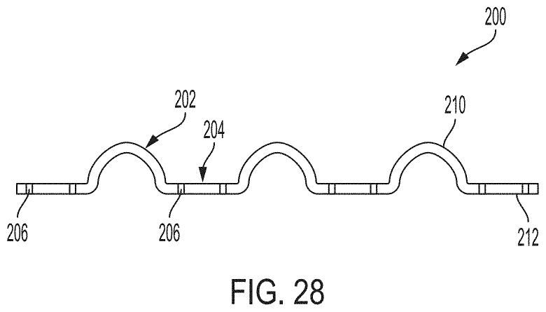

Referring to FIG. 28, if more than one layer is provided in the nonwoven materials 200, the second layer 212 may be nested with the first layer 210. Apertures 206 in the generally planar regions 204 may be formed through the first and second layers 210, 212, or through only one of the layers 210, 212. If recesses are provided, they may be formed in one or more of the layers. In such an instance, the first layer 210 may be joined to the second layer 212 in the three-dimensional features 202 and the generally planar regions 204.

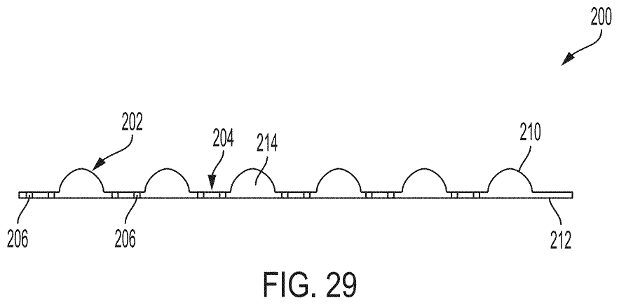

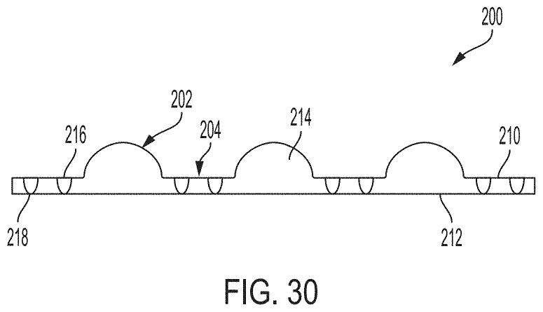

Referring to FIG. 29, the second layer 212 may be generally planar or substantially flat, while the first layer 210 may comprise the three-dimensional features 202 and the generally planar regions 204. The first layer 210 may be joined to the second layer 212 at perimeters of the apertures 206 or in areas proximate to the apertures 206. Voids 214 may be formed intermediate the three-dimensional features 202 and the second layer 212. Referring to FIG. 30, if recesses 216 are provided, distal ends 218 of the recesses (i.e., portions situated most proximate to the second layer) in the first layer 210 may be joined to the second layer 212. Voids 214 may be formed intermediate the three-dimensional features and the second layer 212. Also, recesses may be formed in both layers in some instances.

FIG. 31 is a photograph of a nonwoven material pattern used in options 1, 2, and 4 of Table 1 of the examples. FIG. 32 is a photograph of a nonwoven material pattern used in option 3 of Table 1 of the examples.

### Topsheet Configurations

In some instances, a topsheet for an absorbent article may be formed of three strips with the nonwoven material comprising natural fibers of the present disclosure being the middle strip. The middle strip may comprise one or more layers. A planar, regular, and/or cheaper nonwoven may form the two side strips. In such a fashion, manufacturers can save money by using cheaper nonwoven materials as the side strips, which are mostly covered by leg cuffs, and more expensive nonwoven materials in the middle strip which is most noticeable to consumers and has the most skin contact with a wearer. In one form, the top sheet may only have two components with the cheaper nonwoven material extending the full dimensions of the topsheet and the nonwoven material comprising natural fibers being positioned over the cheaper nonwoven material as a middle longitudinal strip. The nonwoven material comprising natural fibers may also form the entire topsheet.

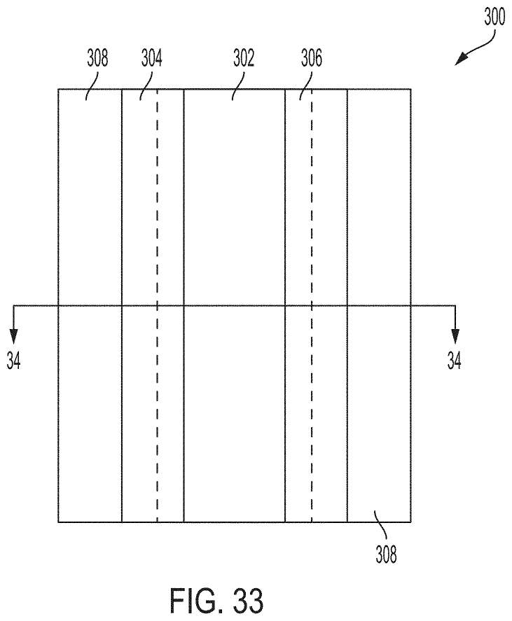

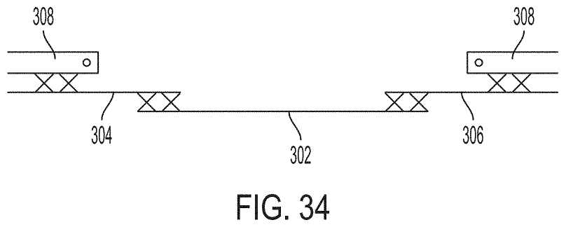

Referring to FIG. 33, a topsheet and leg cuff laminate 300 for an absorbent article is illustrated, wearer-facing surface facing the viewer. A three piece topsheet is provided with a middle strip 302, a first side strip 304, and a second side strip 306. Leg cuffs 308 partially cover the first and second side strips 304, 306. FIG. 34 is a cross-sectional view taken about line 34-34 of FIG. 33. Example bonds or adhesives are illustrated with “XX” in FIG. 34. As can been seen, a portion of the middle strip 302 may be overlapped by a portion of the first side strip 304 and a portion of the second side strip 306. By having the first side strip 304 and the second side strip 306 overlap a portion of the middle strip 302, the middle strip 302 is prevented from, or inhibited from delamination during use. Stated differently, the middle strip 302 is anchored down by the portions of the first side strip 304 and the second side strip 306 to better attach the middle strip 302 and prevent, or inhibit, delamination of the two more layers comprising the middle strip 302. The middle strip 302 may be wider than the first and second side strips 304, 306 or all the strips may have the same width in a direction along a lateral axis of the topsheet laminate 300.

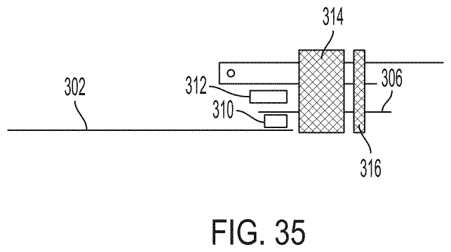

Referring to FIG. 35, a specific bonding construction for the topsheet laminate is now discussed. Only one side of the topsheet laminate is shown since both sides would be the same. The up direction in FIG. 35 is the wearer-facing side. The bonding construction joins the middle strip 302 to the first and second side strips 304 and 306 in portions of the front and back waist regions 12, 16. A tack down bond 312 formed of adhesives is positioned intermediate a garment-facing surface of the leg cuff 308 and a wearer-facing surface of the second side strip 306. A side glue bond 310 is positioned intermediate a garment-facing surface of the second side strip 308 and a wearer-facing surface of the middle strip 302. The glue bond 310 may be applied along a full machine direction length of the topsheet laminate. A first mechanical bond 314 joins both layers of the leg cuff 308 to the second side strip 306. The bond 314, in some instances, may comprise glue instead of or in addition to a mechanical bond. A second mechanical bond 316 joins both layer of the leg cuff 308 to the second side strip 306. The bond 316, in some instances, may comprise glue instead of or in addition to a mechanical bond. This configuration of adhesives and bonding has been found to reduce delamination of the middle strip from the first side strip 304, the second side strip 306, and the absorbent article generally, as well as delamination of two or more layers in the middle strip 302.

### Test Methods

Unless indicated otherwise, all tests described herein are made with samples conditioned at least 24 hours at 23° C.+/−2° C. and 50%+/−10% Relative Humidity (RH).

### Surface Test

The surface bearing area ratio %, average surface roughness (Sa), and root mean square root height (Sq) of the topsheet of an absorbent article are measured using a Digital Light Processing (DLP)-based, structured-light 3D surface topography measurement system. Suitable surface topography measurement system is the GFM Primos Optical Profiler instrument from GFMesstechnik GmbH, WarthestraBe 21, D14513 Teltow/Berlin, Germany. Alternative, yet equivalent, non-contact surface topology profilers having similar principles of measurement and analysis software can also be used, here the GFM Primos and ODSCAD software is exemplified.

The GFM Primos Optical Profiler instrument includes a compact optical measuring sensor based on a digital micro mirror projection, having the following main components:
a) DMD projector with 800×600 direct digital controlled micro-mirrors;
b) CCD camera with high resolution (at least 640×480 pixels);
c) Projection optics adapted to a measuring area of at least 30×40 mm;
d) Recording optics adapted to a measuring area of at least 30×40 mm;
e) A table tripod based on a small hard stone plate;
f) A cold light source (an appropriate unit is the KL 1500 LCD, Schott North America, Inc., Southbridge, Mass.); and
g) A measuring, control, and evaluation computer running surface texture analysis software (suitable software is ODSCAD 6.3 software or equivalent).

The cold-light source is turned on and set to provide a color temperature of at least 2800K.

The image acquisition/analysis software is opened, the “Start Measurement” icon is selected from the ODSCAD 6.3 task bar, and then the “Live Image button” clicked.

The instrument is calibrated according to manufacturer's specifications using calibration plates for lateral (X-Y) and vertical (Z). Such calibration is performed using a rigid solid plate of any non-shiny material having a length of 11 cm, a width of 8 cm, and a height of 1 cm. This plate has a groove or machined channel having a rectangular cross-section, a length of 11 cm, a width of 6.000 mm, and an exact depth of 2.940 mm. This groove is parallel to the plate length direction. After calibration, the instrument must be able to measure the width and depth dimensions of the groove to within ±0.004 mm.

### Specimen Preparation:

To obtain a test specimen, an entire topsheet is removed from an absorbent article. The topsheet can be a single layer or multi-layer laminate. In the case of a multi-layer laminate, the multiple layers are attached either by glue, polymer, ultrasonic or any other known methods. To identify the layers comprising the topsheet, the absorbent article is laid out flat with the body facing side up, and the first uppermost layer is carefully and fully separated from the absorbent article product. The longitudinal length of the first layer is measured and compared to the longitudinal length of the adjacent underlying second layer. If the longitudinal length of the first layer is approximately equal, within 10 mm, of the longitudinal length of the second layer, then first and second layer are part of the topsheet, otherwise, the topsheet is only the first layer. If the first and second layers are approximately equal in length, then this analysis is continued with the third layer, etc., until all the layers of the topsheet have been identified. The lateral widths of the different layers of the topsheet may be different.

The topsheet, as described in the paragraph above, is extracted from the absorbent article by attaching the absorbent article to a flat surface in a planar configuration with the topsheet facing up. Any leg or cuff elastics are severed in order to allow the absorbent article to lie flat. Using scissors, two longitudinal cuts are made through all layers above the absorbent core (i.e., the core wrap) along the edges of the topsheet. Two transversal cuts are made through the same layers at the front and back waist edges of the absorbent article.

The topsheet and any other layers above the absorbent core are then removed without perturbing the topsheet. Freeze spray (e.g., CRC Freeze Spray manufactured by CRC Industries, Inc. 885 Louis Drive, Warminster, Pa. 18974, USA), or equivalent aid may be used to facilitate removal of the layers from the absorbent article. The topsheet is then carefully separated from any underlying layers, such that its longitudinal and lateral extension is maintained to avoid distortion of the apertures. If a distribution layer (e.g., a pulp containing layer) is attached to the topsheet, any residual cellulose fibers are carefully removed with tweezers without modifying the topsheet.

Five replicate specimens, obtained from five substantially similar articles, are prepared for analysis. A topsheet raw material is prepared for testing by extending or activating it under the same process conditions, and to the same extent, as it would be for use on the absorbent article.

The topsheet namely “the specimen” is laid down on a hard flat horizontal surface with the body-facing side upward, i.e., the topsheet skin side being upward. Ensure that the specimen is lying in planar configuration, without being stretched, with the specimen uncovered. The surface to be measured may be lightly sprayed with a very fine white powder spray. Preferably, the spray is NORD-TEST Developer U 89, available from Helling GmbH, Heidgraben, Germany.

A nominal external pressure of 1.86 kPa (0.27 psi) is then applied to the specimen surface. Such nominal external pressure is applied without interfering with the topology profile measurement. Such an external pressure is applied using a transparent, non-shining flat Plexiglas® plate 200 mm by 70 mm and appropriate thickness (approximately 5 mm) to achieve a weight of 83 g. The plate is gently placed on top of the specimen, such that the center point of the Plexiglas® plate is at least 40 mm away from any folds, with the entire plate resting on the specimen. A fold corresponds to a part of the absorbent article where the absorbent article has been folded for packaging purposes.

Two 50 mm×70 mm metal weights each having a mass of 1200 g (approximate thickness of 43 mm) are gently placed on the Plexiglas® plate such that a 70 mm edge of each metal weight is aligned with the 70 mm edges of the Plexiglas® plate. A metal frame having external dimensions of 70 mm×80 mm and interior dimensions of 42 mm×61 mm, and a total weight of 142 g (approximate thickness 6 mm), is positioned in the center of the Plexiglas® plate between the two end weights with the longest sides of the frame aligned with the longest sides of the plate.

If the specimen is smaller than 70×200 mm, or if a large enough area without a fold is not present, or if an area of interest is close to the edges of the specimen and cannot be analyzed with the Plexiglas and weights settings described above, then the X-Y dimensions of the Plexiglas® plate and the added metal weights may be adjusted to reach a nominal external pressure of 1.86 kPa (0.27 psi) while maintaining a minimum 30×40 mm field of view.

The projection head is positioned to be normal to the specimen surface.

The distance between the sample and the projection head is adjusted for best focus. For the Primos Optical Profiler instrument, turn on the button “Pattern” to make a red cross appear on the screen cross and a black cross appears on the sample. Adjust the focus control until the black cross is aligned with the red cross on the screen.

The image brightness is adjusted. For the Primos Optical Profiler instrument, change the aperture on the lens through the hole in the side of the projector head and/or altering the camera “gain” setting on the screen. When the illumination is optimum, the red circle at the bottom of the screen labeled “I.O.” will turn green. Click on the “Measure” button.

The topology of the upper surface of the specimen is measured through the Plexiglas plate over the entire field of view 30 mm×40 mm. It is important to keep the sample still during this time in order to avoid blurring of the captured image. The image should be captured within the 30 seconds following the placement of the Plexiglas plate, metal weights and frame on top of the specimen. We refer to this image as unprocessed image under 1.86 KPa pressure.

The image acquisition procedure described above is also performed on a specimen prior to applying any nominal external pressure to the specimen, in other words without applying the plexiglas plate, metal frame and metal weight. Under such conditions, a topography image of the skin side of the topsheet, without any compression is acquired, and referred to as the unprocessed image without pressure, or 0 Kpa pressure.

After the image has been captured, the X-Y-Z coordinates of every pixel of the 40 mm×30 mm field of view area are recorded. The X direction is the direction parallel to the longest edge of the rectangular field of view, the Y direction is the direction parallel to the shortest edge of the rectangular field of view. The Z direction is the direction perpendicular to the X-Y plane. The X-Y plane is horizontal.

The 3D surface topography image is opened in the surface texture analysis software. The following filtering procedure is then performed on each image: 1) removal of invalid or non-measured points; 2) a 5×5 pixel median filter to remove noise; 3) subtraction of the least square plane to level the surface; 4) a Gaussian filter (according to ISO 16610-61) to smooth the surface with a cut-off wavelength of 15 mm. Such filtering procedure can be applied both to the unprocessed image under 1.86 KPa pressure and the unprocessed image at 0 Kpa pressure.

The processed image, obtained from the unprocessed image via the application of the 4 filters above, is now used to determine the surface bearing area ratio %, the average surface roughness (Sa), and the root mean square root height (Sq).

### Surface Bearing Area Ratio %:

The processed image of the topsheet specimen surface under 1.86 KPa is opened in the surface analysis software, and a reference plane defined as the X-Y plane intercepting the surface topology of the entire field of view (i.e. 30 mm×40 mm) 100 microns below the base X-Y plane. The base X-Y plane height is identified from the Areal Material Ratio curve (Abbott-Firestone), which is the cumulative curve of the surface height distribution histogram versus the range of the surface heights. The base plane height z_{B}, in units of microns, corresponds to the height value at a 2% Areal Material Ratio. Once z_{B} has been determined, the reference plane height z_{R}, in units of microns, is calculated by subtracting 100 microns from the base plane height.

Reference Plane Height (Z_{R})=Base Plane Height (Z_{B})−100 microns

The surface bearing area ratio % is then read from the Areal Material Ratio curve at the reference plane height and recorded to the nearest 1%.

### Average Surface Roughness (Sa) and Root Mean Square Root Height (Sq):

The processed image under 1.86 KPa pressure and the processed image under 0 Kpa pressure are opened in the surface analysis software. As described in ISO 25178-2:2012, the average surface roughness (Sa) and root mean square root height (Sq) are calculated from the processed images. The Sa and Sq values for the 1.86 KPa pressure are recorded to the nearest whole micron, and the Sa and Sq values for the 0 KPa pressure are recorded to the nearest whole micron.

The analysis described above is repeated for each of the five replicate specimen topsheets. The statistical means for the surface bearing area ratio %, average surface roughness (S a), and root mean square root height (Sq) measurements are calculated using all the values recorded for the five replicates and reported.

### Basis Weight Test

Basis weight of the nonwoven materials comprising the natural fibers may be determined by several available techniques, but a simple representative technique involves taking an absorbent article or other consumer product, removing any elastic which may be present and stretching the absorbent article or other consumer product to its full length. A punch die having an area of 45.6 cm² is then used to cut a piece of the nonwoven material (e.g., topsheet, outer cover) from the approximate center of the absorbent article or other consumer product in a location which avoids to the greatest extent possible any adhesive which may be used to fasten the nonwoven material to any other layers which may be present and removing the nonwoven material from other layers (using cryogenic spray, such as Cyto-Freeze, Control Company, Houston, Tex., if needed). The sample is then weighed, and the weight is divided by the area of the punch die yielding the basis weight of the nonwoven material. Results are reported as a mean of 5 samples to the nearest 0.1 g/m² (gsm).

### Aperture Test

Generally planar region dimensions, aperture dimensions, and average minimum distance between apertures measurements are obtained from topsheet specimen images acquired using a flatbed scanner. The scanner is capable of scanning in reflectance mode at a resolution of 600 dpi and 8 bit grayscale (a suitable scanner is an Epson Perfection V750 Pro from Epson America Inc., Long Beach Calif., or equivalent). The scanner is interfaced with a computer running an image analysis program (a suitable program is ImageJ v. 1.52, National Institute of Health, USA, or equivalent). The specimen images are distance calibrated against an acquired image of a ruler certified by NIST. The topsheet specimen is backed with a black glass tile (P/N 11-0050-30, available from HunterLab, Reston, Va., or equivalent) prior to acquiring the image. Identified generally planar regions in the resulting grayscale image are measured, and then the image is converted to a binary image via a threshold gray-level value, enabling the separation of open aperture regions from specimen material regions, and these regions analyzed using the image analysis program.

### Specimen Preparation:

To obtain a test specimen, a topsheet is removed from an absorbent article and prepared as described in the Surface Test.

### Image Acquisition:

The ruler is placed on the scanner bed such that it is oriented parallel to the sides of the scanner glass. An image of the ruler (the calibration image) is acquired in reflectance mode at a resolution of 600 dpi (approximately 23.6 pixels per mm) and in 8-bit grayscale. The calibration image is saved as an uncompressed TIFF format file. After obtaining the calibration image, the ruler is removed from the scanner glass and all specimens are scanned under the same scanning conditions. A topsheet specimen is placed onto the center of the scanner bed, lying flat, with the body facing surface of the specimen facing the scanner's glass surface. The corners and edges of the specimen are secured such that its original longitudinal and lateral extension, as on the article prior to removal, is restored. The specimen is oriented such that the machine direction (MD) and cross direction (CD) of the topsheet specimen layer are aligned parallel with and perpendicular to the sides of the scanner's glass surface and that the resulting specimen image has the MD vertically running from top to bottom. The black glass tile is placed on top of the specimen, the scanner lid is closed, and a scanned image of the entire specimen is acquired. The specimen image is saved as an uncompressed TIFF format file. The remaining four replicate specimens are scanned and saved in like fashion.

### Generally Planar Region Dimension Measurements:

The calibration image (containing the ruler) file is opened in the image analysis program. A linear distance calibration is performed using the imaged ruler. This distance calibration scale is applied to all subsequent specimen images prior to analysis. One specimen image is selected and opened in the image analysis program. The distance scale is set according to the linear distance calibration established using the calibration image. The specimen image is cropped such that is contains one complete individual generally planar region.

The boundary, B, (see e.g., FIGS. 14 and 17) around the individual generally planar region in the specimen image is identified. Using the image analysis software, the identified boundary is outlined, and the area, perimeter, maximum feret diameter (major dimension), and minimum feret diameters (minor dimension) are measured and recorded. The individual generally planar region area is recorded to the nearest 0.01 mm², perimeter and feret diameter (major and minor dimensions), to the nearest 0.01 mm.

### Aperture Dimension Measurements:

The 8-bit grayscale cropped image containing the previously identified individual generally planar region is then converted to a binary image (with “zero” or “black” corresponding to the aperture regions) in the following way: If the histogram of gray level (GL) values (ranging from 0 to 255, one bin with propensity Pᵢ per gray level i) has exactly two local maxima, the threshold gray level value t is defined as that value for which Pₜ₋₁>Pₜ and Pₜ≤Pₜ₊₁. If the histogram has greater than two local maxima, the histogram is iteratively smoothed using a windowed arithmetic mean of size 3, and this smoothing is performed iteratively until exactly two local maxima exist. The threshold gray level value t is defined as that value for which Pₜ₋₁>Pₜ and Pₜ≤Pₜ₊₁. This procedure identifies the gray level (GL) value for the minimum population located between the dark pixel peak of the aperture holes and the lighter pixel peak of the specimen material. If the histogram contains either zero or one local maximum, the method cannot proceed further, and no output parameters are defined. Two morphological operations are then performed on the binary image. First, a closing (a dilation operation, which converts any white background pixel that is touching a black aperture region pixel into a black aperture region pixel thereby adding a layer of pixels around the periphery of the aperture region, followed by an erosion operation, which removes any black aperture region pixel that is touching a white background pixel, thereby removing a layer of pixels around the periphery of the aperture region, iterations=1, pixel count=1) is performed, which removes stray fibers within an aperture hole. Second, an opening (an erosion operation followed by a dilation operation, iterations=1, pixel count=1) is performed, which removes isolated black pixels. The edges of the image are padded during the erosion step to ensure that black boundary pixels are maintained during the operation. Lastly, any remaining voids enclosed within the black aperture regions are filled.

Each of the discrete aperture regions within the identified generally planar region is analyzed using the image analysis software. Any partial apertures along the edges of the image are excluded so that only whole apertures within the identified generally planar region are analyzed. All the individual aperture areas, perimeters, maximum feret diameters (length of the apertures), minimum feret diameters (width of the apertures), and centroid locations are measured and recorded. Individual aperture areas are recorded to the nearest 0.01 mm², aperture perimeters and feret diameters (length and width), to the nearest 0.01 mm. Any apertures with an area less than 0.1 mm² are discarded. The number of remaining apertures within the individual generally planar region is recorded. The number of apertures within the generally planar regions is divided by the area of the generally planar region, and this quotient is recorded as the Aperture Density value to the nearest 0.1 apertures per cm². In addition to these measurements, the Aspect Ratio, defined for each aperture as the quotient of its length divided by its width, is calculated and recorded.

### Average Minimum Distance Between Apertures Measurement:

Using the recorded location of each aperture's centroid within the identified generally planar region, the Euclidian distance between each aperture's centroid to all the other aperture centroids is calculated. For each aperture, the shortest (minimum) distance is then identified and recorded as the nearest neighbor distance. Any spurious distance values that are not representative of apertures within the identified generally planar region are excluded. The arithmetic mean of the recorded nearest neighbor distance values for all the apertures within the identified generally planar region is calculated and reported as the Average Minimum Distance Between Apertures to the nearest 0.1 mm.

The analysis described above is repeated on three different identified generally planar regions from each of the five replicate specimen images for a total of fifteen (15) analyzed generally planar regions. The statistical mean (average) for all the recorded generally planar region dimensions, aperture dimensions, and average distance between apertures measurements are calculated using all the values recorded for the fifteen generally planar regions and reported.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present disclosure have been illustrated and described, it would be obvious to those skilled in the art that various other changes and modifications can be made without departing from the spirit and scope of the present disclosure. It is therefore intended to cover in the appended claims all such changes and modifications that are within the scope of this present disclosure.

What is claimed is:

1. An absorbent article comprising:
a liquid permeable topsheet;
a liquid impermeable backsheet;
an absorbent core positioned at least partially intermediate the topsheet and the backsheet; and
a wearer-facing nonwoven material comprising three-dimensional features and apertures and forming at least a portion of a wearer-facing surface of the topsheet of the absorbent article;
wherein the wearer-facing nonwoven material comprises at least 50% natural fibers, by weight of the wearer-facing nonwoven material; and
a garment-facing nonwoven material forming a garment-facing surface of the topsheet;
wherein the wearer-facing nonwoven material comprises a plurality of generally planar regions that do not overlap with the three-dimensional features;
wherein the three-dimensional features are positioned in zones that do not overlap the generally planar regions;
wherein the three-dimensional features extend outwardly relative to the generally planar regions; and
wherein at least some of the generally planar regions have at least 10 apertures and less than 35 apertures.

2. The absorbent article of claim 1, wherein the garment-facing nonwoven material is generally planar and is joined with the wearer-facing nonwoven material at perimeters of the apertures, wherein the apertures extend through the wearer-facing nonwoven material and the garment-facing nonwoven material, and wherein voids are defined intermediate the three-dimensional features of the wearer-facing nonwoven material and the garment-facing nonwoven material.

3. The absorbent article of claim 1, wherein the garment-facing nonwoven material is nested with the wearer-facing nonwoven material.

4. The absorbent article of claim 1, wherein the garment-facing nonwoven material comprises synthetic fibers and is substantially free of natural fibers.

5. The absorbent article of claim 1, wherein the apertures have an average aperture length in the range of about 0.4 mm to about 1.1 mm.

6. The absorbent article of claim 1, wherein the wearer-facing nonwoven material comprises more natural fibers than the garment facing nonwoven material.

7. The absorbent article of claim 1, wherein the three-dimensional features comprise protrusions.

8. The absorbent article of claim 1, wherein a basis weight of the wearer-facing nonwoven material is in the range of about 15 gsm to about 45 gsm, according to the Basis Weight Test.

9. The absorbent article of claim 1, wherein the natural fibers comprise hydrophilic cotton fibers.

10. The absorbent article of claim 1, wherein the natural fibers comprise hydrophobic cotton fibers.

11. The absorbent article of claim 1, wherein the wearer-facing nonwoven material is hydroentangled and comprises synthetic fibers.

12. The absorbent article of claim 1, wherein the generally planar regions have an average area in the range of about 20 mm² to about 100 mm², and wherein the generally planar regions have an average perimeter in the range of about 10 mm to about 75 mm.

13. The absorbent article of claim 1, wherein an average minimum distance between two of the closest apertures in a generally planar region is in the range of about 0.5 mm to about 4 mm.

14. The absorbent article of claim 1, wherein the generally planar regions have an average major dimension in the range of about 5 mm to about 20 mm, and wherein the generally planar regions have an average minor dimension in the range of about 3 mm to about 15 mm.

15. The absorbent article of claim 1, wherein the generally planar regions are substantially continuous.

16. The absorbent article of claim 1, wherein the three-dimensional features are substantially continuous.

17. The absorbent article of claim 1, wherein the apertures have an average area in the range of about 0.15 mm² to about 0.6 mm², and wherein the apertures have an average perimeter in the range of about 1 mm to about 2.8.

18. The absorbent article of claim 1, wherein the natural fibers comprise plant-based fibers, bio-based polyolefin fibers, bio-based polyester fibers, or combinations thereof.

19. The absorbent article of claim 1, wherein the generally planar regions have an total aperture area in the range of about 20% to about 80%.

20. The absorbent article of claim 1, wherein the zones are surrounded on at least two sides by the generally planar regions.

21. The absorbent article of claim 1, wherein the generally planar regions are surrounded on at least two sides by the zones.
